(19) United States
(12) Patent Application Publication
(10) Pub. No.: US 2015/0226751 A1
(43) Pub. Date: Aug. 13, 2015

(54) Methods for Diagnosing Mood Disorders

(71) Applicant: Nunzio Pomara, Rye Brook, NY (US)

(72) Inventor: Nunzio Pomara, Rye Brook, NY (US)

(21) Appl. No.: 14/314,145

(22) Filed: Jun. 25, 2014

Related U.S. Application Data
(63) Continuation of application No. 13/523,063, filed on Jun. 14, 2012, now abandoned.
(60) Provisional application No. 61/520,721, filed on Jun. 14, 2011.

(51) Int. Cl. G01N 33/68

(52) U.S. Cl. CPC G01N 33/6893; G01N 2333/4709; G01N 2800/304; G01N 2800/50

(57) ABSTRACT

The present invention relates to methods of diagnosing, prognosing or treating diseases or disorders in which elevated levels of Aβ protein, including Aβ₁₋₄₂ are prevalent. In particular, the present invention relates to methods of diagnosing, prognosing or treating a mood disorder such as a major or minor depressive disorder or dysthymia attributed to reduced levels of Aβ protein, including Aβ₁₋₄₂, found particularly in body fluids including whole blood, blood cells, serum, plasma, urine and CSF. The invention also relates to the treatment of these disorders and methods of identifying subjects who may respond positively to such treatment by administering an agent that either prevents production of Aβ, prevents aggregation of Aβ fibrils, that increases the degradation or clearance of Aβ or that prevents or interferes with Abeta-induced neurotoxicity.

### FIELD OF THE INVENTION

The present invention relates to the identification of decreased levels of amyloid beta protein, for example, Aβ₁₋₄₂, in certain biological fluids such as cerebrospinal fluid in some subjects suffering from a mood disorder such as major depressive disorder.

### BACKGROUND

In the majority of major depressive disorders, little is known about a link between changes at the cellular or molecular level and nervous system structure and function. The paucity of detectable neurologic defects distinguishes major depressive disorders from neurological disorders where manifestations of anatomical and biochemical changes have been identified. Thus, the identification and characterization of cellular or molecular causative defects is desirable for improved treatment of major depressive disorders.

Depressive disorders come in different forms. A major depressive episode is manifested by a combination of symptoms that interfere with the ability to work, study, sleep, eat, and enjoy once pleasurable activities. The DSM-IV diagnostic criteria can be found in Tables I, II and III. Such a disabling episode of depression may occur only once but more commonly occurs several times in a lifetime. A minor depressive disorder is characterized by one or more periods of depressive symptoms that are identical to those found in major depression in duration, but which involve fewer symptoms and less impairment. Dysthymic disorder is a less serious form of depression and involves long-term, chronic symptoms that do not disable, but keep one from functioning well or from feeling good. Many people with dysthymia also experience major depressive episodes at some time in their lives. Bipolar disorder, also called manic-depressive illness is not nearly as prevalent as other forms of depressive disorders and is characterized by cycling mood changes, including severe highs (mania) and lows (depression).

Some types of depression appear to have a genetic component associated with their occurrence, suggesting that a biological vulnerability can be inherited. This appears to be the case with bipolar disorder. Furthermore, major depression seems to occur generation after generation. However, it can also occur in people who have no family history of depression.

Major depressive disorder is prevalent in a large number of elderly patients, resulting in a significant increase in the number of suicides in this population. The risk factors for late-life depression include female gender, unmarried status, having stressful life events and lack of a social support network. Major depressive disorder is characterized by any of a number of symptoms, including persistent sadness or anxiety, or feelings of emptiness, hopelessness, pessimism, guilt, worthlessness, or helplessness. There may also be a loss of interest or pleasure in hobbies and activities that were once enjoyed. Individuals with major depressive disorder may also experience decreased energy, or fatigue, or may have difficulty concentrating, remembering, or making decisions. They may also experience insomnia, early-morning awakening, or oversleeping, appetite and/or weight loss or overeating and weight gain. They may have thoughts of death or suicide and suicide attempts. They may also experience restlessness, irritability, and may exhibit persistent physical symptoms that do not respond to treatment, such as headaches, digestive disorders, and chronic pain.

Major mood disorders are also associated with many other deleterious health related effects and the costs with disability and premature death represent an economic burden of $43 billion annually in the United States alone. Rates of depression co-occurring with other medical conditions are as follows: myocardial infarction: 20-40%, Parkinson's disease: 40%, Alzheimer's disease: 30-35%, stroke: 25-50%, cancer: 3-50%, HIV/AIDS: 10-20%, rheumatoid arthritis: 12%, diabetes mellitus: 14-18%, chronic pain: 30%, disabling tinnitus: 60%, end-stage renal disease: 5-22% and spinal cord injury: 37% (Goldman et al. J Gen Intern Med 1999, 14, 569-580; Wyatt, et al., 1995, Soc Psychiatry Psychiatr Epidemiolm 1995; 30:213-219). Despite the devastating impact of these disorders on the lives of millions, there is still uncertainty about the differential diagnosis of depression in the presence of these disorders (Goldman et al. 1999, J Gen Med 14, 569-80; Schatzberg, J Clin Psychiatry, 1998; 59, suppl 6:5-12; Goodwin and Jamison, 1990 Manic-depressive illness, New York, Oxford University Press).

Current therapies can be categorized into the following major classes of agents: mood stabilizers: lithium, divalproex, carbamazepine, lamotrigine; antidepressants: tricyclic antidepressants (eg. Desipramine, chlorimipramine, nortriptyline), selective serotonin reuptake inhibitors (SSRIs including fluoxetine (Prozac), sertraltrine (Zoloft), paroxetine (Paxil), fluvoxamine (Luvox), and citalopram (Celexa)), MAOIs, bupropion (Wellbutrin), venlafaxine (Effexor), and mirtazapine (Remeron); and atypical antipsychotic agents: Clozapine, Olanzapine, Risperidone. However, the cellular and molecular basis for the efficacy of currently used mood-stabilizing and mortality-lowering agents remains to be fully elucidated (Manji et al., J Clin Psychiatry, 1999; 60:27-39). A significant number of patients respond poorly to existing therapies such as lithium, while many others are helped but continue to suffer significant morbidity (Chou, J Clin Psychopharmacol 1991; 11:3-21). The recognition of the significant morbidity and mortality of the severe mood disorders, as well as the growing appreciation that a significant percentage of patients respond poorly to existing treatments, has made the task of developing new therapeutic agents that work quickly, potently, specifically, and with fewer side effects one of major public health importance (Bebchuk et al. Arch Gen Psychiatry 2000 57, 95-7).

Hence it would be highly desirable to measure a substance or substances in samples of whole blood, blood cells, serum, plasma, urine or cerebrospinal fluid (CSF) that would lead to a positive diagnosis of a mood disorder such as major depressive disorder or that would help to predict whether an individual is prone to developing such disorder. For example, the identification of proteins and/or the nucleic acids encoding proteins that are associated with the onset and progression of a major depressive disorder would be desirable for the effective diagnosis, prognosis and treatment of afflicted individuals. Appropriate steps may then be taken to treat early on with existing therapies. Alternatively, if one could demonstrate an association between a certain substance, such as a protein, in the blood of a depressed individual, it may be possible to utilize this information to develop new modes of therapy to prevent or treat such depressive disorders.

Given that the CSF bathes the brain, changes in its protein composition may reveal alterations in CNS protein expression pattern causatively or diagnostically linked to the disease. Alternatively, if reasonable amounts of proteins associated with the major depressive disorder are secreted or released into body fluids by diseased tissue in the living patient at the onset and/or during progression of the disease, such information may be utilized for early diagnosis and expedite a treatment strategy. In many cases these alterations will be independent of the genetic makeup of the individual and rather directly related to a set of molecular and cellular alterations that contribute to the pathogenic phenotype.

Cerebrospinal fluid (CSF) Aβ42 is now considered a biomarker of Alzheimer's Disease (AD), and AD subjects show lower CSF levels as compared to controls that inversely correlate with the presence of fibrillar Aβ in the brain, as measured by PET amyloid tracers. Other important CSF biomarkers of AD and MCI are total (T) tau and hyperphosphorolylated (P) tau, markers of neuronal degeneration and neurofibrillary tangles, respectively, whose levels are found to be greater in CSF for AD sufferers than controls.

CSF Aβ and tau levels have also been studied in conjunction with major depression (MDD) in elderly individuals. Gudmundsson et al. (1997; 2000) compared elderly women with MDD to controls with no depressive symptoms. None of these subjects had dementia. The MDD group had higher CSF Aβ42 levels than controls, suggesting that subjects with MDD may have lower brain Aβ deposition than controls.

However, recent in vivo PET studies, using PIB and FDDNP, have shown increased Aβ brain deposition in subjects with depression as opposed to controls. For instance, a recent study (e.g., Marano et al., Neuropsychopharmacology, 2010; 35:S78-S187) that looked at currently depressed subjects with MDD and no mild cognitive impairment (MCI), using PET-PIB images before an antidepressant course of treatment, found that depressed subjects (n=6) showed increased brain Aβ deposition as compared to age-matched controls (n=6). In contrast, neither T nor P tau CSF levels were found to differ across elderly subjects with MDD and controls.

Therefore, a need exists to identify sensitive and specific biomarkers for the diagnosis of mood disorders such as major and minor depressive disorders and to assess a subject's risk for developing such disorders. There is also a need for a biomarker to assess the severity and to predict the outcome of a major or minor depressive disorder in living subjects or to assess whether a particular therapy is efficacious in treating a subject having such disorders. More importantly, the need exists for identification of a biomarker substance that may be found in a body fluid that can be obtained with minimally invasiveness procedures for early and specific diagnosis of a mood disorder such as major depressive disorder. Additionally, there is a clear need for new therapeutic agents for major depressive disorders that work quickly, potently, specifically, and with fewer side effects.

### SUMMARY OF THE INVENTION

The present invention relates to methods of screening, diagnosing or prognosing a mood disorder such as a major or minor depressive disorder or dysthymia in a subject. The invention further relates to identifying a subject at risk for developing a mood disorder such as a major or minor depressive disorder or for monitoring the effect of therapy administered to a subject suffering from a mood disorder such as a major or minor depressive disorder. More particularly, the methods of the invention are based on the previously unidentified finding of a decrease in or reduced concentrations of cerebrospinal fluid (CSF) amyloid β protein (in particular, Aβ₁₋₄₂) in elderly individuals suffering from a major depressive disorder. The invention further relates to methods of screening a subject for elevated levels of this protein in bodily fluids, in particular blood and/or plasma and/or urine, as a means of determining whether that subject has, or is prone to developing, a mood disorder such as a major or minor depressive disorder or dysthymia. This protein may also be used as a means of assessing the effectiveness of therapy in subjects being treated for a mood disorder such as a major or minor depressive disorder or dysthymia. Thus, such procedure for screening or diagnosing subjects with such conditions is a minimally invasive procedure that allows for rapid and sensitive screening. Furthermore, the association of decreased levels of Aβ protein or fragments thereof, such as Aβ₁₋₄₂, with depressive disorders, allows for the identification and development of new therapeutic regimens directed towards lowering the levels of Aβ proteins or fragments thereof in the brain, or concomitantly increasing levels of Aβ proteins or fragments in the cerebrospinal fluid (CSF) for treating such depressive disorders. As such, the present invention provides methods for identifying subjects suffering from a mood disorder such as a major or minor depressive disorder or dysthymia who may be responsive to treatment with therapeutic agents that may lower brain levels of or concentrations of amyloid beta. In addition, because Aβ₁₋₄₀ is known to be neurotoxic (like Aβ₁₋₄₂), lowering the levels of both Aβ₁₋₄₂ as well as, Aβ₁₋₄₀ in the brain may be beneficial in treating depressive disorders and depressive symptoms. It may be necessary to lower the levels of Aβ proteins or peptide fragments, such as Aβ₁₋₄₀ and Aβ₁₋₄₂ to a level in the brain that poses no risk for development of depressive disorders or symptoms. The invention further relates to pharmaceutical compositions containing an agent that reduces brain amyloid beta levels in the brain, particularly Aβ₁₋₄₂, which may be achieved by such agent acting to prevent the production or generation of amyloid beta, by preventing the aggregation of amyloid beta, by increasing the degradation of amyloid beta, by increasing the clearance of amyloid beta from the brain, by preventing the neurotoxicity associated with Aβ or by facilitating central and peripheral metabolism and clearance of amyloid beta. The invention also relates to screening methods that aid in the identification of novel agents that function through any one of the above-noted mechanisms for use in the treatment of a major or minor depressive disorder. The invention also relates to the inhibition of formation of potentially neurotoxic oligomeric forms of Aβ, especially Aβ₁₋₄₂.

In a first aspect of the invention provides a method of screening, diagnosis or prognosis of a mood disorder such as a major or minor depressive disorder in a subject comprising:
a) collecting a biological test sample from said subject;
b) determining the level of or concentration of amyloid beta in the biological test sample; and
c) comparing the level of or concentration of amyloid beta in the test sample with the level of or concentration of amyloid beta in one or more persons free from a mood disorder such as major or minor depressive disorder, or with a previously determined reference range for amyloid beta established from subjects free of a mood disorder such as major or minor depressive disorder.

In particular embodiments, the methods are used to identify a subject at risk for developing a major or minor depressive disorder, or for monitoring the effect of therapy administered to a subject having a major or minor depressive disorder. In some embodiments, the test sample is selected from the group consisting of whole blood, blood cells, serum, plasma, urine and CSF. In particular embodiments, reduced levels or concentrations of amyloid beta in the cerebrospinal fluid of a subject correlates with the presence of a major depressive disorder. In some embodiments, the Aβ is Aβ₁₋₄₂. In some embodiments, amyloid beta is measured by a quantitative method such as an immunological or biochemical assay specific for amyloid beta. In yet other particular embodiments, amyloid beta is measured by an enzyme-linked immunosorbent assay (ELISA), a Western blot assay, a Northern blot assay, and a Southern blot assay. In particular embodiments, the amyloid beta is amyloid β₁₋₄₀ (Aβ₄₀) or amyloid β₁₋₄₂ (Aβ₄₂) or fragments thereof.

In some embodiments, the quantitative method comprises testing at least one aliquot of the test sample, comprising the steps of:
a. contacting/incubating the aliquot with an antibody that is immunospecific for amyloid beta; and
b. quantitatively measuring any binding that has occurred between the antibody and the test sample.

In some embodiments, the antibody is a monoclonal or polyclonal antibody specific for amyloid beta, in particular, amyloid beta₁₋₄₀ or amyloid beta₁₋₄₂.

In other embodiments, the step of quantitatively measuring comprises testing a plurality of aliquots with a plurality of antibodies for the quantitative detection of amyloid beta.

In some particular embodiments, this aspect of the invention provides a method of diagnosing a mood disorder, such as major depression, minor depression or dysthymia in a subject comprising:

a) collecting a biological test sample selected from the group consisting of whole blood, serum, plasma, urine and cerebrospinal fluid from said subject;

b) determining the concentration of or level of or concentration of amyloid beta present in the biological test sample;

c) comparing the concentration of or level of or concentration of amyloid beta in the test sample with the concentration of or level of or concentration of amyloid beta in a biological test sample obtained from one or more persons free from a mood disorder such as major depression, minor depression or dysthymia, or with a previously determined reference range for amyloid beta established from subjects free of a mood disorder such as major depression, minor depression or dysthymia, and

d) diagnosing a mood disorder such as major depression, minor depression or dysthymia in the subject if the level of or concentration of amyloid beta is lower in the biological test sample from the subject than the level of or concentration of amyloid beta in one or more persons free from a mood disorder such as major depression, minor depression or dysthymia, or with a previously determined reference range for amyloid beta established from subjects free of a mood disorder such as major depression, minor depression or dysthymia.

In a particular embodiment, the amyloid beta is amyloid β₁₋₄₀ or amyloid β₁₋₄₂ or fragments thereof. In some embodiments the biological fluid is cerebrospinal fluid, whole blood, plasma, serum or urine, and in some embodiments the subject is elderly. In many embodiments the subject is free of cognitive impairment or is not suffering from dementia such as that experienced with mild cognitive impairment or Alzheimer's Disease.

In a second aspect, the invention provides a method of treating and/or preventing a mood disorder such as a major or minor depressive disorder or dysthymia comprising administering a therapeutically effective amount of an agent that reduces intracellular and/or extracellular amyloid beta levels in the brain or increases amyloid beta levels in a biological fluid such as the cerebrospinal fluid (CSF). In addition, the invention provides a method of treating or preventing a mood disorder such as a major or minor depressive disorder or dysthymia comprising administering an agent that prevents or interferes with Abeta-induced neurotoxicity.

In a particular embodiment, reducing the amyloid beta level is achieved by a preventing the production or generation of amyloid beta, by preventing the aggregation of amyloid beta fibrils and deposition in cerebral parenchymal tissues and blood vessels, by preventing the formation of oligomeric forms of Aβ, Aβ 1-40 or 1-42, by increasing the degradation of amyloid beta, by increasing the clearance of amyloid beta from the brain, or by facilitating the central and/or peripheral metabolism and clearance of amyloid beta. In a particular embodiment, the amyloid beta is amyloid β₁₋₄₀ or amyloid β₁₋₄₂ or fragments thereof. In another particular embodiment, the reduction in amyloid beta levels results in reduced neurotoxicity.

In certain embodiments of the present invention, an agent/compound that reduces brain amyloid beta can be administered in combination therapy with at least one other (a second) therapeutic agent. In a preferred embodiment, a composition comprising an agent/compound that reduces brain amyloid beta is administered concurrently with a second therapeutic agent, which can be part of the same composition as or in a different composition from that comprising the agent/compound that reduces brain amyloid beta including oligomeric forms of this peptide. In another embodiment, a composition comprising a compound that reduces brain amyloid beta is administered prior or subsequent to administration of a second therapeutic agent. As many of the disorders for which the compounds of the invention are useful in treating are chronic, in one embodiment combination therapy involves alternating between administering a composition comprising a compound that reduces brain amyloid beta and a composition comprising a second therapeutic agent, e.g., to minimize the toxicity associated with a particular drug. The agent/compound that reduces brain amyloid beta or and/or second therapeutic agent can be administered for, e.g., one month, three months, six months, a year, or for more extended periods, or on an alternate or intermittent schedule. In certain embodiments, when a compound that reduces brain amyloid beta is administered concurrently with a second therapeutic agent that potentially produces adverse side effects including, but not limited to, toxicity, the second therapeutic agent can advantageously be administered at a dose that falls below the threshold at which the adverse side is elicited. In another particular embodiment, the second drug that is used is effective at treating a mood disorder such as a major or minor depressive disorder or dysthymia. In yet another particular embodiment, the second drug is administered concurrently, prior to, or after the agent that reduces brain amyloid beta levels. In yet another particular embodiment, the second drug may be a Selective Serotonin Reuptake Inhibitors (SSRI), a tricyclic antidepressant, or a monoamine oxidase (MAO) inhibitor. In a more particular embodiment, the Selective Serotonin Reuptake Inhibitor may be citalopram, escitalopram HBr, fluvoxamine, paroxetine, fluoxetine, or sertraline. In a more particular embodiment, the tricyclic antidepressant may be amitriptyline, desipramine, doxepin, protriptyline, trimipramine

or nortriptyline. In a more particular embodiment, the monoamine oxidase inhibitor may be phenelzine or tranylcypromine. In a more particular embodiment the second drug is a drug that acts as an inhibitor of neuronal reuptake of both serotonin (5-HT) and norepinephrine (NE). In a more particular embodiment, the inhibitor of neuronal reuptake of both serotonin and norepinephrine is venlafaxine or duloxetine.

In another particular embodiment, the method comprises administering an agent that may interfere with the neurotoxic effects of amyloid beta on neurons or other brain cells, or on synaptic connections, which could lead to the development of, or may contribute to, the pathophysiology of a mood disorder such as a major or minor depressive disorder or dysthymia in humans.

A third aspect of the invention provides a pharmaceutical composition for the treatment of a mood disorder such as a major or minor depressive disorder or dysthymia. The pharmaceutical composition may contain an agent that prevents the production or generation of amyloid beta and a pharmaceutically acceptable carrier, or an agent that prevents aggregation of amyloid beta fibrils and deposition in cerebral parenchymal tissues or blood vessels, or prevents the formation of oligomeric forms of Aβ, Aβ 1-40 or 1-42, or an agent that increases the degradation of amyloid beta and a pharmaceutically acceptable carrier, or an agent that increases the clearance of amyloid beta from the brain and a pharmaceutically acceptable carrier, or an agent that facilitates the metabolism of amyloid beta and a pharmaceutically acceptable carrier. The pharmaceutical composition may be formulated for delivery by any suitable route such as intravenous, intramuscular, oral, subcutaneous, intrathecal, intracranial or intraventricular. In a particular embodiment, the amyloid beta is amyloid β₁₋₄₀ or amyloid β₁₋₄₂.

A fourth aspect of the invention provides a method of identifying a subject at risk for developing a mood disorder such as a major or a minor depressive disorder or dysthymia comprising:
a) collecting a biological test sample from the subject;
b) determining the concentration of or levels of amyloid beta in the biological test sample; and
c) comparing the concentration of or level of or concentration of amyloid beta in the biological test sample with the concentration of or level of or concentration of amyloid beta in a biological test sample obtained from one or more persons free from a major or a minor depressive disorder, or with a previously determined reference range for amyloid beta established from subjects free of a major or a minor depressive disorder.

In a particular embodiment, the amyloid beta is amyloid β₁₋₄₀ or amyloid β₁₋₄₂ or fragments thereof. In another particular embodiment, the test sample is one or more of whole blood, blood cells, serum, plasma, urine and cerebrospinal fluid (CSF). In some embodiments the biological fluid is cerebrospinal fluid, whole blood, plasma, serum or urine, and in some embodiments the subject is elderly. In many embodiments the subject is free of cognitive impairment or is not suffering from dementia such as may be the result of mild cognitive impairment or Alzheimer's Disease.

A fifth aspect of the invention provides a diagnostic test for assessing a subject's risk of developing or having a mood disorder such as a major or minor depressive disorder or dysthymia or for determining a response to therapy for the mood disorder, comprising the following steps:
a) collecting one tissue or cellular sample from said subject;
b) measuring the level of or concentration of amyloid beta in one tissue or cellular sample; and
c) comparing the levels of amyloid beta with a range of predetermined values for amyloid beta, said values having been determined as falling within a reference range of amyloid beta.

In a particular embodiment, the amyloid beta is amyloid β₁₋₄₀ or amyloid β₁₋₄₂ or fragments thereof. In another particular embodiment, the test sample is selected from the group consisting of whole blood, blood cells, serum, plasma, urine and CSF. In some instances, the subject is elderly.

A sixth aspect of the invention provides a method of measuring the effectiveness of an agent for treating a subject having a mood disorder such as a major or minor depressive disorder or dysthymia, comprising the steps of:
a) determining the level of or concentration of amyloid beta in a biological test sample obtained from the subject;
b) administering an amount of said agent to the patient;
c) repeating step a) using a subsequently-collected biological sample obtained from the subject;
d) comparing the level of or concentration of amyloid beta determined in step a) with the level of or concentration of amyloid beta determined in step c), wherein the effectiveness of the pharmaceutical composition is monitored by detecting an increase in the level of or concentration of amyloid beta in the subsequently-collected biological sample compared with the biological sample from step a).

In a particular embodiment, the amyloid beta is amyloid β₁₋₄₀ or amyloid β₁₋₄₂ or fragments thereof. In another particular embodiment, the test sample is selected from the group consisting of whole blood, blood cells, serum, urine, CSF and plasma. In some instances, the subject is elderly.

A seventh aspect of the invention provides a method for screening for agents that inhibit the production or generation of Aβ, or inhibit/prevent the aggregation of amyloid beta fibrils and deposition in cerebral parenchymal tissues or blood vessels, or by preventing the formation of oligomeric forms of Aβ, Aβ₁₋₄₀ or Aβ₁₋₄₂ or blocking their effect, or enhance the degradation or clearance of Aβ comprising the steps of:
a) combining amyloid beta protein with an antibody specific for amyloid beta protein in a first sample;
b) combining a test agent, amyloid beta protein and an antibody specific for amyloid beta protein in a second sample;
c) comparing the difference in binding to amyloid beta between the two samples; and
wherein a change or difference in binding to amyloid beta between the two samples indicates the presence of a test agent capable of binding to amyloid beta.

In one embodiment the method comprises assessing a change in amyloid beta structure to determine any conformational/structural changes or changes in aggregation e.g. formation of amyloid beta fibrils. Methods for determining these changes are known to one skilled in the art and encompass standard biochemical and/or immunochemical assays. In another particular embodiment, the amyloid beta is Aβ₄₀, Aβ₄₂, or fragments thereof.

In an eighth aspect, the present invention provides a method of identifying a subject suffering from a mood disorder such as major or minor depression or dysthymia that may be successfully treated by an agent that affects amyloid beta levels comprising:
a) collecting a biological test sample selected from the group consisting of whole blood, serum, plasma, urine and cerebrospinal fluid from said subject;
b) determining the level of or concentration of amyloid beta present in the biological test sample;
c) comparing the level of or concentration of amyloid beta in the test sample with the level of or concentration of amyloid beta in one or more persons free from a mood disorder such as major depression, minor depression or dysthymia, or with a previously determined reference range for amyloid beta established from subjects free of a mood disorder such as major depression, minor depression or dysthymia, and
d) determining whether the level of or concentration of amyloid beta is lower in the biological test sample from the subject than the level of or concentration of amyloid beta in one or more persons free from a mood disorder such as major depression, minor depression or dysthymia, or with a previously determined reference range for amyloid beta established from subjects free of a mood disorder such as major depression, minor depression or dysthymia.

In a particular embodiment, the amyloid beta is amyloid β₁₋₄₀ or amyloid β₁₋₄₂ or fragments thereof. In another particular embodiment, the biological test sample is one or more of whole blood, blood cells, serum, plasma, urine and cerebrospinal fluid (CSF), and in some embodiments the subject is elderly. In many embodiments the subject is free of cognitive impairment or is not suffering from dementia such as that experienced with mild cognitive impairment or Alzheimer's Disease.

### BRIEF DESCRIPTION OF THE DRAWINGS

FIG. 1 demonstrates reduced levels of Abeta measured in cerebrospinal fluid obtained from elderly patients suffering from major depressive disorder compared to controls.

FIG. 2 demonstrates elevated levels of F2-isoprostanes measured in cerebrospinal fluid obtained from elderly patients suffering from major depressive disorder compared to controls. The level of F2-isoprostanes is a measure of oxidative stress.

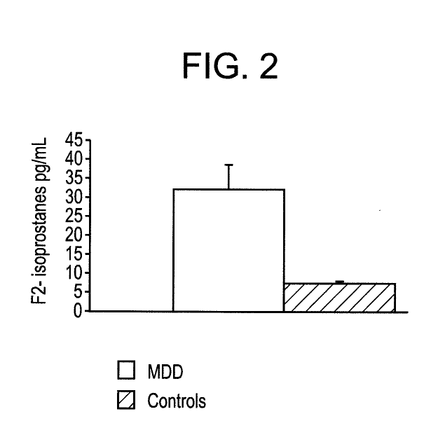

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference in their entirety.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. PHames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

### DEFINITIONS

The terms used herein have the meanings recognized and known to those of skill in the art, however, for convenience and completeness, particular terms and their meanings are set forth below. Mood disorder is the term given for a group of diagnoses in the Diagnostic and Statistical Manual of Mental Disorders (DSM IV TR) classification system where a disturbance in the person's mood is hypothesized to be the main underlying feature. The classification is known as mood (affective) disorders in ICD 10. Two groups of mood disorders are broadly recognized; the division is based on whether the person has ever had a manic or hypomanic episode. Thus, there are depressive disorders, of which the best known and most researched is major depressive disorder (MDD) commonly called clinical depression or major depression, and bipolar disorder (BD), formerly known as “manic depression” and described by intermittent periods of manic and depressed episodes.

Depressive disorders include “Major depressive disorder (MDD)” commonly called major depression, unipolar depression, or clinical depression, where a person has two or more major depressive episodes. Depression without periods of mania is sometimes referred to as unipolar depression because the mood remains at one emotional state or “pole”. Diagnosticians recognize several subtypes or course specifiers: Atypical depression (AD) is characterized by mood reactivity (paradoxical anhedonia) and positivity, significant weight gain or increased appetite (“comfort eating”), excessive sleep or somnolence (hypersomnia), a sensation of heaviness in limbs known as leaden paralysis, and significant social impairment as a consequence of hypersensitivity to perceived interpersonal rejection. Difficulties in measuring this subtype have led to questions of its validity and prevalence. Melancholic depression is characterized by a loss of pleasure (anhedonia) in most or all activities, a failure of reactivity to pleasurable stimuli, a quality of depressed mood more pronounced than that of grief or loss, a worsening of symptoms in the morning hours, early morning waking, psychomotor retardation, excessive weight loss (not to be confused with anorexia nervosa), or excessive guilt. Psychotic major depression (PMD), or simply psychotic depression, is the term for a major depressive episode, particularly of melancholic nature, where the patient experiences psychotic symptoms such as delusions or, less commonly, hallucinations. These are most commonly mood-congruent (content coincident with depressive themes). Catatonic depression is a rare and severe form of major depression involving disturbances of motor behavior and other symptoms. Here the person is mute and almost stuporose, and either immobile or exhibits purposeless or even bizarre movements. Catatonic symptoms also occur in schizophrenia, a manic episode, or be due to neuroleptic malignant syndrome. Postpartum depression (PPD) is listed as a course specifier in DSM-IV-TR; it refers to the intense, sustained and sometimes disabling depression experienced by women after giving birth. Postpartum depression, which has incidence rate of 10-15%, typically sets in within three months of labor, and lasts as long as three months. Seasonal affective disorder (SAD), also known as “winter depression” or “winter blues”, is a specifier. Some people have a seasonal pattern, with depressive episodes coming on in the autumn or winter, and resolving in spring. The diagnosis is made if at least two episodes have occurred in colder months with none at other times over a two-year period or longer.

“Dysthymia,” which is a chronic, different mood disturbance where a person reports a low mood almost daily over a span of at least two years. The symptoms are not as severe as those for major depression, although people with dysthymia are vulnerable to secondary episodes of major depression (sometimes referred to as double depression). Depressive Disorder Not Otherwise Specified (DD-NOS) is designated by the code 311 for depressive disorders that are impairing but do not fit any of the officially specified diagnoses. According to the DSM-IV, DD-NOS encompasses “any depressive disorder that does not meet the criteria for a specific disorder.” It includes the research diagnoses of recurrent brief depression, and minor depressive disorder listed below. Recurrent brief depression (RBD), distinguished from major depressive disorder primarily by differences in duration. People with RBD have depressive episodes about once per month, with individual episodes lasting less than two weeks and typically less than 2-3 days. Diagnosis of RBD requires that the episodes occur over the span of at least one year and, in female patients, independently of the menstrual cycle.^{[} People with clinical depression can develop RBD, and vice versa, and both illnesses have similar risks. “Minor depressive disorder,” or simply minor depression, which refers to a depression that does not meet full criteria for major depression but in which at least two symptoms are present for two weeks.

“Bipolar disorder” (BD), a mood disorder formerly known as “manic depression” and described by alternating periods of mania and depression (and in some cases rapid cycling, mixed states, and psychotic symptoms). Subtypes include: Bipolar I is distinguished by the presence or history of one or more manic episodes or mixed episodes with or without major depressive episodes. A depressive episode is not required for the diagnosis of Bipolar I disorder, but depressive episodes are often part of the course of the illness. Cyclothymia is a different form of bipolar disorder, consisting of recurrent hypomanic and dysthymic episodes, but no full manic episodes or full major depressive episodes. Bipolar Disorder Not Otherwise Specified (BD-NOS), sometimes called “sub-threshold”bipolar, indicates that the patient suffers from some symptoms in the bipolar spectrum (e.g. manic and depressive symptoms) but does not fully qualify for any of the three formal bipolar DSM-IV diagnoses mentioned above. It is estimated that roughly one percent of the adult population suffers from bipolar I, roughly one percent of the adult population suffers from bipolar II or cyclothymia, and somewhere between two and five percent suffer from “sub-threshold” forms of bipolar disorder.

Substance-induced mood disorders refers to a mood disorder that can be classified as substance-induced if its etiology can be traced to the direct physiologic effects of a psychoactive drug or other chemical substance, or if the development of the mood disorder occurred contemporaneously with substance intoxication or withdrawal. Alternately, an individual may have a mood disorder coexisting with a substance abuse disorder. Substance-induced mood disorders can have features of a manic, hypomanic, mixed, or depressive episode. Most substances can induce a variety of mood disorders. For example, stimulants such as amphetamine (Adderall, Dexedrine; “Speed”), methamphetamine (Desoxyn; “Meth”, “Crank”, “Crystal”, etc), and cocaine (“Coke”, “Crack”, etc) can cause manic, hypomanic, mixed, and depressive episodes. Alcohol-induced mood disorders includes major depressive disorder occurring in heavy drinkers and those with alcoholism. Controversy has previously surrounded whether those who abused alcohol and developed depression were self-medicating their pre-existing depression, but recent research has concluded that, while this may be true in some cases, alcohol misuse directly causes the development of depression in a significant number of heavy drinkers. High rates of suicide also occur in those who have alcohol-related problems. It is usually possible to differentiate between alcohol-related depression and depression which is not related to alcohol intake by taking a careful history of the patient. Depression and other mental health problems associated with alcohol misuse may be due to distortion of brain chemistry, as they tend to improve on their own after a period of abstinence.

Benzodiazepine-induced mood disorders may be associated with long term use of benzodiazepines which have a similar effect on the brain as alcohol and are also associated with depression. Major depressive disorder can also develop as a result of chronic use of benzodiazepines or as part of a protracted withdrawal syndrome. Benzodiazepines are a class of medication which are commonly used to treat insomnia, anxiety and muscular spasms. As with alcohol, the effects of benzodiazepine on neurochemistry, such as decreased levels of serotonin and norepinephrine, are believed to be responsible for the increased depression. Major depressive disorder may also occur as part of the benzodiazepine withdrawal syndrome. In a long-term follow-up study of patients dependent on benzodiazepines, 10 people (20%) had taken drug overdoses while on chronic benzodiazepine medication despite only two people ever having had any pre-existing depressive disorder. A year after a gradual withdrawal program, no patients had taken any further overdoses. Depression resulting from withdrawal from benzodiazepines usually subsides after a few months but in some cases may persist for 6-12 months.

“Anxiety disorder” is a blanket term covering several different forms of abnormal and pathological fear and anxiety which only came under the aegis of psychiatry at the very end of the 19th century. Current psychiatric diagnostic criteria recognize a wide variety of anxiety disorders. Recent surveys have found that as many as 18% of Americans may be affected by one or more of them.

Anxiety disorders are often debilitating chronic conditions, which can be present from an early age or begin suddenly after a triggering event. They are prone to flare up at times of high stress and are frequently accompanied by physiological symptoms such as headache, sweating, muscle spasms, palpitations, and hypertension, which in some cases lead to fatigue or even exhaustion.

Although in casual discourse the words anxiety and fear are often used interchangeably, in clinical usage, they have distinct meanings; anxiety is defined as an unpleasant emotional state for which the cause is either not readily identified or perceived to be uncontrollable or unavoidable, whereas fear is an emotional and physiological response to a recognized external threat. The term anxiety disorder, however, includes fears (phobias) as well as anxieties. Anxiety disorders are often comorbid with other mental disorders, particularly clinical depression, which may occur in as many as 60% of people with anxiety disorders. The fact that there is considerable overlap between symptoms of anxiety and depression, and that the same environmental triggers can provoke symptoms in either condition, may help to explain this high rate of comorbidity.

Types of anxiety disorders include generalized anxiety disorder, panic disorder, phobias, agoraphobia, social anxiety disorder, obsessive-compulsive disorder, post-traumatic stress disorder, and separation anxiety disorder.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to prevent, and preferably reduce by at least about 25 percent, more preferably by at least 50 percent, most preferably by at least 90 percent, a clinically significant change in a feature of pathology such as for example, elevated blood pressure, fever, or white cell count, as may attend its presence and activity. As related to the present invention, the term may also mean an amount sufficient to ameliorate or reverse one or more symptoms associated with the depressive order being treated. In particular, a “therapeutically effective amount” of the treatment may result in amelioration, reduction or elimination of at least one of the following symptoms: persistent sadness or anxiety, feelings of emptiness, hopelessness, pessimism, guilt, worthlessness, helplessness, a loss of interest or pleasure in hobbies and activities that were once enjoyed, decreased energy, or fatigue, difficulty concentrating, remembering, or making decisions, insomnia, early-morning awakening, or oversleeping, appetite and/or weight loss or overeating and weight gain, thoughts of death or suicide and suicide attempts, restlessness, irritability, and persistent physical symptoms that do not respond to treatment, such as headaches, digestive disorders, and chronic pain.

“Treatment” or “treating” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted.

“Subject” or “patient” refers to a mammal, preferably a human, in need of treatment for a condition, disorder or disease.

An “antibody” is any immunoglobulin, including antibodies and fragments thereof, that binds a specific epitope. Such an antibody that binds a specific epitope is said to be “immunospecific”. The term encompasses “polyclonal”, “monoclonal”, and “chimeric” antibodies, the last mentioned described in further detail in U.S. Pat. Nos. 4,816,397 and 4,816,567. Commonly used carriers that are chemically coupled to peptides include bovine or chicken serum albumin, thyroglobulin, and other carriers known to those skilled in the art. The coupled peptide is then used to immunize the animal (e.g, a mouse, rat or rabbit). The “chimeric antibody” refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397) The antibody may be a human or a humanized antibody. The antibody may be a single chain antibody. (See, e.g., Curiel et al., U.S. Pat. No. 5,910,486 and U.S. Pat. No. 6,028,059). The antibody may be prepared in, but not limited to, mice, rats, rabbits, goats, sheep, swine, dogs, cats, or horses. As used herein, the term “single-chain antibody” refers to a polypeptide comprising a V_{H} region and a V_{L} region in polypeptide linkage, generally linked via a spacer peptide (e.g., [Gly-Gly-Gly-Gly-Ser] and which may comprise additional amino acid sequences at the amino- and/or carboxy-termini. For example, a single-chain antibody may comprise a tether segment for linking to the encoding polynucleotide. As an example, a scFv (single chain fragment variable) is a single-chain antibody. Single-chain antibodies are generally proteins consisting of one or more polypeptide segments of at least 10 contiguous amino acids substantially encoded by genes of the immunoglobulin superfamily (e.g., see The Immunoglobulin Gene Superfamily, A. F. Williams and A. N. Barclay, in Immunoglobulin Genes, T. Honjo, F. W. Alt, and T. H. Rabbitts, eds., (1989) Academic Press: San Diego, Calif., pp. 361-387, which is incorporated herein by reference), most frequently encoded by a rodent, non-human primate, avian, porcine, bovine, ovine, goat, or human heavy chain or light chain gene sequence. A functional single-chain antibody generally contains a sufficient portion of an immunoglobulin superfamily gene product so as to retain the property of binding to a specific target molecule, typically a receptor or antigen (epitope).

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

“Screening” or “Diagnosis” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

The essential features of a “major depressive episode” or “major depression” is a period of at least 2 weeks during which there is either a depressed mood or the loss of interest or pleasure in nearly all activities. The individual must also experience at least four additional symptoms drawn from a list that includes changes in appetite or weight, sleep, and psychomotor activity; decreased energy; feelings of worthlessness or guilt; difficulty thinking, concentrating, or making decisions; or recurrent thoughts of death or suicidal ideation, plans or attempts. To be considered a major depressive episode, a symptom must have clearly worsened compared with the person's preepisode status. The symptoms must persist for most of the day, nearly every day, for at least 2 consecutive weeks. The episode must be accompanied by clinically significant distress or impairment in social, occupational or other important areas of functioning (Diagnostic and Statistical Manual of Mental Disorders 4^{th} Ed. DSM-IV, Pub. American Psychiatric Association, Washington, D.C.; p. 320, 327, 344-345). A “major depressive disorder” generally refers to a single or recurrent Major Depressive Episode which is not better accounted for by Schizophrenia, Delusional Disorder, or Psychotic Disorder Not Otherwise Specified, and also there has never been a Manic Episode, a Mixed Episode or a Hypomanic Episode (Diagnostic and Statistical Manual of Mental Disorders 4^{th} Ed. DSM-IV, Pub. American Psychiatric Association, Washington, D.C.; pp. 344-345). The diagnosis is generally based on evaluation by a qualified physician, generally a psychiatrist or by a psychologist. A “minor depressive disorder”, also referred to as “dysthymia”, has the characteristics of a major depressive disorder but presents itself without the intensity or severity of the symptoms associated with a “major depressive disorder”. “Late Life Major Depression”, referred to as “LLMD” or “late-onset depression” refers to depression, for example, the major and minor depressive disorders and depressive episodes described above, that occurs in a subject at about 60 years of age or older. The “risk factors” for depression include female gender, unmarried status, having stressful life events and lack of a social support network. Major depressive disorder is characterized by any of a number of symptoms, including persistent sadness or anxiety, or feelings of emptiness, hopelessness, pessimism, guilt, worthlessness, or helplessness.

“Amyloid” describes various types of protein aggregations that share specific traits when examined microscopically. Amyloid is typically identified by a change in the fluorescence intensity of planar aromatic dyes such as Thioflavin T or Congo Red. This is generally attributed to the environmental change as these dyes intercolate between β-strands. The amyloid fold is characterized by a cross-β sheet quaternary structure, that is, a monomeric unit contributes a β strand to a β sheet which spans across more than one molecule. While amyloid is usually identified using fluorescent dyes, stain polarimetry, circular dichroism, or FTIR (all indirect measurements), the “gold-standard” test to see if a structure is amyloid is by placing a sample in an X-ray diffraction beam; there are two characteristic scattering bands produced at 4 and 10 angstroms each, corresponding to the interstrand distances in the β sheet structure. The amyloid protein disclosed in the present application refers to amyloid β, as described below.

“Amyloid beta”, “Aβ”, “β-amyloid” or “amyloid beta peptide” is a physiological product normally released from the amyloid beta protein precursor (βAPP or APP) through β and γ secretase cleavage and consists of two 40 and 42 amino acid peptides, usually abbreviated as Aβ₄₀ and Aβ₄₂, respectively (Selkoe, J. Clin. Invest. (1992); 110:1375-1381). The 42 amino acid amyloid beta peptide (Aβ₄₂) is more hydrophobic & “sticky” (and hence aggregates more readily) than the 40 amino acid amyloid beta peptide (Aβ₄₀), and as such may play a greater role in the pathogenesis of Alzheimer's disease, due to its increased tendency to form insoluble fibrils and increased neurotoxicity. Thus, under certain circumstances, as in Alzheimer's disease (AD), brain levels of these peptides increase dramatically, which can lead to the oligomerization of the peptides and eventually to the formation of insoluble fibrillar aggregates, which deposit in senile plaques. In the present application, “amyloid β₄₀” is used interchangeably with “Aβ₄₀”, Aβ₄₀ and Aβ₁₋₄₀, and “amyloid β₄₂” is used interchangeably with “Aβ₄₂”, Aβ₄₂ and Aβ₁₋₄₂. The nucleic acid and amino acid sequences for amyloid beta precursor protein and Aβ₄₀ and Aβ₄₂ are found in SEQ ID NOS: 1, 2, 3, 4 and 5. SEQ ID NO: 1 is the nucleic acid sequence encoding human amyloid beta precursor protein; SEQ ID NO: 2 is the nucleic acid encoding human Aβ₄₀ peptide; SEQ ID NO: 3 is the amino acid sequence of human Aβ₄₀ peptide; SEQ ID NO: 4 is the nucleic acid encoding human Aβ₄₂, SEQ ID NO: 5 is the amino acid sequence of human Aβ₄₂ peptide.

“Fragment” refers to either a protein or polypeptide comprising an amino acid sequence of at least 4 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the amino acid sequence of a parent protein or polypeptide, or a nucleic acid comprising a nucleotide sequence of at least 10 base pairs (preferably at least 20 base pairs, at least 30 base pairs, at least 40 base pairs, at least 50 base pairs, at least 50 base pairs, at least 100 base pairs, at least 200 base pairs) of the nucleotide sequence of the parent nucleic acid. Any given fragment may or may not possess a functional activity of the parent nucleic acid or protein or polypeptide.

“Combination therapy” refers to the use of the agents of the present invention with other active agents or treatment modalities, in the manner of the present invention for treatment of major depressive disorders. These other agents or treatments may include drugs such as other antidepressants including those that are standardly used to treat various depressive disorders. The agents of the invention may also be combined with corticosteroids, non-steroidal anti-inflammatory compounds, or other agents useful in treating or alleviating pain. The combined use of the agents of the present invention with these other therapies or treatment modalities may be concurrent, or the two treatments may be divided up such that the agent of the present invention may be given prior to or after the other therapy or treatment modality.

“Slow release formulation” refers to a formulation designed to release a therapeutically effective amount of a drug or other active agent such as a polypeptide or a synthetic compound over an extended period of time, with the result being a reduction in the number of treatments necessary to achieve the desired therapeutic effect. In the matter of the present invention, a slow release formulation would decrease the number of treatments necessary to achieve the desired effect in terms of amelioration of, reduction in, or reversal of at least one symptom of the mood disorder such as a major or minor depressive disorder or dysthymia.

“Surrogate biomarker” or “biomarker” as used herein, refers to a molecule, the existence and levels of which are causally connected to a complex biological process, and reliably captures the state of said process. Furthermore, a surrogate biomarker, to be of practical importance, must be present in samples that can be obtained from individuals without endangering their physical integrity or well-being, preferentially from biological fluids such as blood, urine, saliva or tears.

An “antidepressant” is a medication used primarily in the treatment of clinical depression. Modern antidepressants are not stimulants and are not generally addictive. They also are not thought to produce tolerance, although sudden withdrawal may produce adverse effects. Antidepressants create little if any immediate change in mood and require between several days and several weeks to take effect. There are several different compounds that act as antidepressants. These have different mechanisms of action as well as different chemical structures, many of which are described herein.

“Selective serotonin reuptake inhibitors” or “SSRIs”, as described herein, are a class of antidepressants. They act within the brain to increase the amount of the neurotransmitter serotonin (5-hydroxytryptamine or 5-HT) in the synaptic gap by inhibiting its re-uptake.

“Tricyclic antidepressants”, as described herein, are a class of antidepressant drugs named after the drugs' molecular structure, which contains three rings of atoms. The term ‘tricyclic antidepressant’ is sometimes abbreviated to TCA. Most tricylic antidepressants work by inhibiting the re-uptake of the neurotransmitters norepinephrine and serotonin by nerve cells. They are not considered addictive and have many fewer side-effects and restrictions than the MAOIs.

“Monoamine Oxidase Inhibitors” or “MAOIs”, as described herein, are a class of antidepressant drugs that act to inhibit the activity of the enzyme monoamine oxidase, an enzyme that is found in many parts of the body. In the brain, monoamine oxidase destroys neurotransmitters, such as norepinephrine and serotonin. MAO inhibitors, by limiting the activity of monoamine oxidase, block the breakdown of those neurotransmitters. They work more quickly than the tricyclics, but they have more severe side effects and require a change in diet.

A “subject at risk for developing a major or minor depressive disorder” or a “subject at risk for developing a major or minor depressive episode”, as described herein, refers to a subject who is predisposed to development of a major depressive or minor depressive disorder as described in the present application, by virtue of a past history of such episodes. The occurrence of such episodes may or may not be related to a particular identifiable stressor, which leads to development of such depressive disorder.

“Monitoring the effect of therapy administered to a subject” as used herein refers to a situation whereby a subject is being treated for a major or minor depressive disorder through use of at least one of the drugs described herein for clinical depression, and it is desirable to determine whether there are any benefits to such treatment. The effects of such therapy may be determined by evaluating the subject using standard procedures for such evaluation by a trained physician, preferably a psychiatrist or clinical psychologist. Well known rating scales for depression are used, such as the Hamilton scale, whereby a score of 15 or greater is necessary to establish the presence of significant depressive symptoms associated with major depression that may warrant pharmacological intervention. A 50% reduction in the Hamilton score is generally considered to be therapeutically responsive. A patient having a score of 7 or less is considered to be in remission.

“Abnormal levels of amyloid beta”, especially Aβ1-42 as defined herein are established by determining the amount of amyloid beta in the brains or body fluid, such as, urine, CSF, blood (whole blood, blood cells or plasma or serum) of normal patients who do not suffer from a major depressive disorder, and comparing these levels with that of patients who suffer from a mood disorder such as a major or minor depressive disorder or dysthymia. For example, as shown herein, normal elderly control patients show a level of or concentration of plasma amyloid beta, in particular, Aβ42, of 8.1 pg/ml, whereas depressed elderly patients show a value of plasma amyloid beta, in particular, Aβ42, 14.3 pg/ml. The difference between the normal (non-depressed) and depressed population of patients should be significantly different at the 95% confidence limit. However, it is recognized by the present invention that any reduction in plasma amyloid beta is good, particularly, Aβ1-42, and thus, even a modest lowering (although perhaps not statistically significant when compared to normals) will have an impact on a mood disorder such as a major or minor depressive disorder or dysthymia or episode.

Amyloid beta levels are said to be lowered, reduced or decreased or the concentration of amyloid beta in a biological test sample is said to be lowered, reduced, or decreased when the amyloid beta is present in an absolute number that is less than the amount present in a control sample, such as, for instance 5%, 10%, 20%, 25%, 30%, 40%, 50%, 60%, 70%, 80% or more less than the concentration or amount of amyloid beta found in a control sample such as a biological test sample.

By “elderly” is meant at that a subject is at least about 55 years old, at least about 60 years old, at least about 62 years old, at least about 65 years old or at least about 70 years old.

### General Description

Major depressive episode has been reported to be associated with higher rates of neuroradiological abnormalities including deep white matter lesions, cognitive impairment, and an increased risk for Alzheimer's disease (AD). However, the biological mechanisms underlying the development of symptoms and for the higher rates of structural brain abnormalities and for the increased risk for Alzheimer's disease (AD) are not known. The present invention relates to the finding of elevated plasma amyloid beta peptide (Aβ₄₂) levels in non-demented, non-Alzheimer's disease (AD) patients suffering from late life major depression (LLMD), which were also associated with greater white matter abnormalities. While the Aβ₄₂ peptide has been shown to play a major role in the pathogenesis in AD and elevated plasma Aβ₄₂ levels have been associated with increased risk for AD and white matter abnormalities, there have been no previous reports of elevated plasma Aβ₄₂ levels in non-demented, non-AD elderly patients suffering from late life major depression. The finding of elevated plasma Aβ₄₂ in LLMD could serve as a potential biological marker for the development of depressive symptoms and for an increased risk for brain abnormalities and development of a major depressive episode/disorder. Furthermore, since Aβ₄₂ has been shown to be neurotoxic, the finding of elevated levels of Aβ₄₂ in the peripheral circulation of patients suffering from depressive episodes/disorders may explain why these patients are more prone to development of Alzheimer's disease. Although patients suffering from Alzheimer's disease exhibit high levels of aggregated, insoluble Aβ fibrils deposited in the brain, which are believed to be neurotoxic and possibly causative of the dementia and cognitive impairment in these patients, the inventors of the present application propose that circulating levels of Aβ, especially Aβ₄₂, and in all likelihood other soluble amyloid beta fragments that are capable of crossing the blood brain barrier (BBB), also result in neurotoxicity and disruption of serotoninergic, noradrenergic and dopaminergic neurons (Gonzalo-Ruiz, et al., J. Chemical Neuroanatomy 2003; 26: 153-169), which have been implicated in depression even though such fragments and soluble forms of amyloid beta need not necessarily be deposited in the brain. Thus, it is believed that circulating oligomeric forms of Aβ₄₂ can cross the blood brain barrier and be neurotoxic and may result in, or be conducive to, the initiation of a major depressive disorder/episode. Furthermore, the findings presented herein may explain why patients suffering from a major depressive episode/disorder are prone to future development of Alzheimer's disease, cognitive dysfunction and dementia.

A number of studies have found an association between cognitive impairment, AD or dementia, and depressed mood or late-life major depression (LLMD). In a meta-analysis of case-control and prospective longitudinal studies of depression and dementia (Jorm, Australian and New Zealand Journal of Psychiatry. 2001; 35:776-781), it was concluded that depressed individuals are, on average, nearly twice as likely to develop dementia, often in the form of AD, relative to non-depressed controls. For example, one prospective longitudinal study (Devanand, et al., Arch Gen Psychiatry 1996; 53:175-182) found that the presence of depressed mood at baseline was significantly associated with a moderately increased likelihood of incident dementia. Since 93% of the individuals who developed dementia during the follow-up also met the National Institute of Neurological and Communicative Disorders and Stroke-Alzheimer's Disease and Related Disorders Association criteria (NINCDS/ADRDA) for possible or probable AD, the increased likelihood applied predominantly to AD. Excluding individuals who had a form of dementia other than Alzheimer's Disease did not alter the results. Furthermore, limiting the analyses to include only individuals who were cognitively intact at baseline did not attenuate the relationship between LLMD and incident AD. Another well-designed study (Wilson, et al., Neurology 2002; 59: 364-370) examined the association of depressive symptoms and likelihood for the development of clinical AD or cognitive decline annually over a 7-year period in 821 (n=554 women) older adults who were not demented at baseline. Proportional hazards models were used to determine whether CES-D scores were related to the likelihood of developing clinical AD. Results revealed that for each 1 point increase in the depressive symptoms, as measured by a 10-item form of the CES-D scale, the relative risk (Odds Ratio) of AD increased by an average of 19%, and annual decline on a global cognitive measure increased by an average of 24%. Similarly, Yaffe et al. (Yaffe, et al., Arch Gen Psychiatry 1999; 56:425-430) found an inverse relationship between number of depressive symptoms and cognitive change in women who were 65 and older and cognitively intact at baseline. Specifically, in analyses accounting for education, age, health status, baseline cognitive scores, and a variety of health behaviors (i.e. tobacco use and alcohol consumption), women with more depressive symptoms showed significantly greater decline over four-years on the Trails B (attention, sequencing, visual scanning, and mental flexibility), Digit Symbol (attention, psychomotor speed, and perceptual organization), and a modified version of the MMSE test (global cognitive functioning) relative to women with few or no depressive symptoms. Likewise, in another prospective longitudinal analysis of depressed and non-depressed adults aged 59 to 71 who were cognitively intact at baseline (Paterniti, et al., British J Psychiatry 2002; 181:406-410), participants at high risk for clinical depression, as measured by high scores on the CES-D, exhibited significantly greater decline on the MMSE over four-years relative to participants who did not have high scores on the CES-D.

Further support for the relatively strong association between LLMD or depressive symptoms and AD is provided by two recent post-mortem studies. For example, in a longitudinal study that included 80 initially non-demented elderly followed through autopsy (mean age at baseline=80.7, sd=9.3 years; baseline CDR=0), greater depressive symptoms were associated with increased likelihood of a dementia diagnosis (Galvin, et al., Arch Neurol 2005; 62:758-765). AD was histologically confirmed in 79% of the individuals who developed dementia. Interestingly, the number of cerebral infarcts did not differ between those with dementia and those who remained nondemented through follow-up. Sweet et al.'s post-mortem findings (Sweet, et al., Neuropsychopharm 2004; 29: 2242-2250), like those of Galvin et al.'s results (Galvin, et al., Arch Neurol 2005; 62:758-765), also indicated that AD accounts for most cases of dementia. In this study, all of the subjects had their first depressive episode after the age of 60, met the criteria for LLMD based on the Structured Clinical Interview for DSM-IV (SCID), and were all cognitively intact at baseline (Sweet, et al., Neuropsychopharm 2004; 29: 2242-2250). Comprehensive neuropsychological assessments were done annually, and autopsies were performed on 10 subjects. The time interval from the onset of depression to death was 57 months (median). Seven out of 10 individuals developed dementia during the longitudinal follow-up and 6 (86%) out of 7 of these individuals had a pathological diagnosis of AD. It is also noteworthy that 3 out of the four individuals who had significant vascular pathology and all 3 individuals with Lewy bodies also met pathologic criteria for AD. Additionally, only the AD pathology was significantly associated with dementia (p=0.03). To summarize, both of these post-mortem studies confirm the predominance of AD pathology in elderly individuals with depressed moods or late onset LLMD who develop dementia. Overall, the results derived from the aforementioned case studies, prospective longitudinal studies, and post-mortem studies indicated that a substantial number of these individuals developed dementia or exhibited significant cognitive decline within a few years.

The relationship between depression and cognitive decline or AD is not fully understood. Some have argued that depression is associated with reversible cognitive decline. Other studies suggest that LLMD-associated cognitive deficits, particularly executive function, may not necessarily resolve with successful antidepressant treatment and may be associated with subsequent conversion to AD (Alexopoulos, J Clin Psychiatry 2003; 64 Suppl 14:18-23; Kalayam, et al., Arch Gen Psychiatry 1999; 56:713-718). Additionally, there are also emerging data from a variety of neuroimaging techniques consistent with the presence of structural and functional brain abnormalities in individuals with LLMD (Krishnan, et al., Biol Psychiatry 2004; 55:390-397; Kumar, et al., Psychiatry Res 2004; 130:131-140).

The findings from the aforementioned studies indicated that not every individual with LLMD would go on to develop AD during longitudinal follow-up. For example, in Sweet and colleagues (Sweet, et al., Neuropsychopharm 2004; 29: 2242-2250) post-mortem study, 3 out of the 10 subjects did not develop AD. Accordingly, it would be useful to find a biological marker that would help to identify those individuals with late-onset LLMD who may have prodromal AD. Results from the studies presented herein suggest that elevated plasma Aβ₄₂ and greater reductions over time may be useful biomarkers for identifying with late-onset LLMD who may have prodromal AD. The Aβ peptides, especially the 42 amino acid form (Aβ₄₂), produced through proteolytic cleavage of the amyloid precursor protein (APP) have been demonstrated to have powerful neurotoxic effects. Elevated brain levels have been implicated in the pathogenesis of AD (Cleary, et al., Nat Neurosci 2005; 8:79-84; Jhoo, et al., Behav Brain Res 2004; 155:185-196; Walsh, et al., Protein Pept Lett 2004; 11:1-16). Results from longitudinal studies involving relatively large numbers of cognitively intact elderly (Mayeux, et al., Ann Neurol 1999; 46:412-416; Mayeux, et al., Neurology 2003; 61:1185-1190), also indicated that elevated plasma Aβ₄₂ levels may be a significant risk factor for the development of AD in the general population. Furthermore, prior longitudinal research has also revealed a significant association between Aβ₄₂ and cognitive decline (Pomara, et al., Am J Geriatr Psychiatry 2005; 13:914-917). Specifically, higher baseline Aβ₄₂ levels and greater reductions in Aβ₄₂ were significantly associated with a greater decline in MMSE scores over four years.

There is also evidence that platelet activation, which is believed to be a major source of plasma Aβ (Chen, et al., Biochem Biophys Res Commun 1995; 213: 96-103; Li, et al., Blood 1994; 84:133-142) and other APP products, might be increased in individuals with depression (Lagrhrissi-Thode, et al., Biol Psychiatry 1997; 42:290-295; Markovitz, et al., Am J Psychiatry 2000; 157:1006-1008; Musselman, et al., Am J Psychiatry 1996; 153:1313-1317; Piletz, et al., J Psychiatric Res 2000; 34:397-404; Pollock, et al., J Clin Psychopharmacol 2000; 20:137-140). While the findings noted above raised the possibility that elevated plasma Aβ levels could occur in individuals with LLMD and contribute to the increased susceptibility to AD and brain abnormalities associated with this disorder, the data presented herein demonstrate that there is no relationship between platelet activation and Aβ levels in patients having LLMD. Similar findings were also reported by Olsson, A. et al. (Olsson, et al. Dement Geriatr Cogn Disord 2003; 16(2):93-7).

The present invention demonstrates that Aβ levels in elderly individuals with LLMD differed from nondepressed controls and whether they were associated with age of onset of first depressive episode, antidepressant treatment. Since elevated Aβ₄₂ is associated with cognitive decline and incident AD, the finding of decreased Aβ₄₂ in some body fluids in elderly depressives provides a useful approach for further investigation of the utility of Aβ₄₂ to identify individuals with LLMD who might develop subsequent cognitive decline and AD. Furthermore, the presence of decreased levels of Aβ42 in some biological fluids provides a non-invasive means of assessing an individual's risk of developing late onset depression and depressive symptoms, and provides a causative biological factor for the development of depressive symptoms and disorders.

Accordingly, the present invention relates in general to identifying decreased levels of amyloid beta in tissues and/or body fluids of patients suffering from a mood disorder such as a major depressive disorder. More particularly, the invention relates to identifying decreased levels of amyloid beta in a body fluid, more particularly the cerebrospinal fluid, from non-demented, non-AD, elderly depressed patients.

In addition, the present invention provides for amyloid beta as a biomarker of clinical depression. In accordance with the present invention, the amyloid beta protein or fragments thereof, can be obtained using minimally invasive procedures. In a particular aspect, the invention provides for methods of screening, diagnosis or prognosis of a major depressive disorder in a subject, or for identifying a subject at risk for developing a major depressive disorder, or for monitoring the effect of therapy administered to a subject having a major depressive disorder. In a particular embodiment, the method comprises the steps of:
a. collecting a biological test sample from said subject;
b. analyzing said test sample for the presence of amyloid beta levels; and
c. comparing the level of or concentration of amyloid beta in the test sample with the level of or concentration of amyloid beta in one or more persons free from a major depressive disorder, or with a previously determined reference range for amyloid beta established from subjects free of major depressive disorder.

While the amyloid beta or fragment thereof may be measured in any bodily tissue sample from the subject, it is desirable to perform the measurement from a sample of bodily fluid, such as, but not limited to whole blood, blood cells, serum, plasma, urine and cerebrospinal fluid (CSF). In this manner, the sample may be obtained by non-invasive or minimally invasive procedures. Thus, the determination of elevated or decreased levels of amyloid beta in the sample from a patient who potentially has a major depressive disorder or who is prone to development of a major depressive disorder can be made and such measurement can be used as a surrogate marker for determination of a major depressive disorder. Accordingly, in one particular embodiment, a decreased level or concentration of amyloid beta in a subject correlates with the presence of a major depressive disorder. In another particular embodiment, the amyloid beta is A β₄₀ or A β₄₂ and the elevation of amyloid beta is the result of an increase in production or generation of A β₄₀ or A β₄₂ as determined by measurement of A β₄₀ or A β₄₂ in a 24 hour urine sample. The level of or concentration of amyloid beta may be measured by standard immunological and/or biochemical assays known to one skilled in the art, such as enzyme-linked immunosorbent assays (ELISA), Western blot assays, Northern blot assays, and Southern blot assays.

The methods of the present invention provide for use of an antibody that binds to (is specific for) amyloid beta, Aβ₄₀, Aβ₄₂ or fragments thereof and may be a monoclonal or polyclonal antibody specific for amyloid beta, in particular, amyloid β₁₋₄₀ or amyloid β₁₋₄₂. In a particular embodiment, the step of quantitatively measuring comprises testing a plurality of aliquots with a plurality of antibodies for the quantitative detection of amyloid beta.

In a particular embodiment, the amyloid beta is amyloid β₁₋₄₀ or amyloid β₁₋₄₂ or fragments thereof. In another particular embodiment, the test sample is selected from the group consisting of whole blood, blood cells, serum, urine, CSF and plasma.

### Screening Methods for Measuring Amyloid Beta Levels

Antibodies to Amyloid beta for Therapeutic or Diagnostic Use

According to the present invention, amyloid beta, including a β₄₀ and a β₄₂, as produced by a recombinant source, or through chemical synthesis, or isolated from natural sources; and derivatives, analogs and fragments thereof, including fusion proteins, may be used as an immunogen to generate antibodies that recognize amyloid beta, as exemplified below. Such antibodies include but are not limited to polyclonal, monoclonal, chimeric including humanized chimeric, single chain, Fab fragments, and a Fab expression library. The anti-amyloid beta antibodies, for example, of the invention may be cross reactive, that is, they may recognize an amyloid beta derived from a different source. Polyclonal antibodies have greater likelihood of cross reactivity. Alternatively, an antibody of the invention may be specific for a single form of amyloid beta, such as the human amyloid beta proteins amyloid β₄₀ or amyloid β₄₂, or a fragment of a human amyloid beta protein.

Various procedures known in the art may be used for the production of polyclonal antibodies to amyloid beta or derivatives, analogs or fragments thereof. For the production of an antibody, various host animals can be immunized by injection with amyloid beta, or a derivative (e.g., or fusion protein) thereof, including but not limited to rabbits, mice, rats, sheep, goats, etc. In one embodiment, amyloid beta or a fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward amyloid beta, or analogs, derivatives or fragments thereof, any technique that provides for the production of antibody molecules by continuous cell lines in culture may be used. These include but are not limited to the hybridoma technique originally developed by Kohler and Milstein (Nature, 1975; 256:495-497), as well as the trioma technique, the human B-cell hybridoma technique (Kozbor et al., Immunology Today, 1983; 4:72; Cote et al., Proc. Natl. Acad. Sci. U.S.A., 1983; 80:2026-2030), and the EBV-hybridoma technique to produce human monoclonal antibodies (Cole et al., in Monoclonal Antibodies and Cancer Therapy, Alan R. Liss, Inc., 1985; pp. 77-96 (1985)). In an additional embodiment of the invention, monoclonal antibodies can be produced in germ-free animals utilizing recent technology (PCT/US90/02545). In fact, according to the invention, techniques developed for the production of “chimeric antibodies” (Morrison et al., J. Bacteriol., 1984; 159:870; Neuberger et al., Nature, 1984; 312:604-608; Takeda et al., Nature, 1985; 314:452-454) by splicing the genes from a mouse antibody molecule specific for amyloid beta together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies (U.S. Pat. Nos. 5,476,786 and 5,132,405 to Huston; U.S. Pat. No. 4,946,778) can be adapted to produce e.g., amyloid beta-specific single chain antibodies. An additional embodiment of the invention utilizes the techniques described for the construction of Fab expression libraries (Huse et al., Science, 1989; 246:1275-1281) to allow rapid and easy identification of monoclonal Fab fragments with the desired specificity for amyloid beta, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays, etc. Once the antibody is produced, the antibody may be employed in the assays noted above to screen bodily tissues or fluids for the presence of amyloid beta or fragments thereof. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of amyloid beta, one may assay generated hybridomas for a product which binds to the amyloid beta or fragment containing such epitope and choose those which do not cross-react with amyloid beta. For selection of an antibody specific to amyloid beta from a particular source, one can select on the basis of positive binding with amyloid beta expressed by or isolated from that specific source.

The foregoing antibodies can be used in methods known in the art relating to the localization and activity of the amyloid beta, e.g., for Western blotting, imaging amyloid beta in situ, measuring levels thereof in appropriate physiological samples, etc. using any of the detection techniques mentioned herein or known in the art. The standard techniques known in the art for immunoassays are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, J. Clin. Chem. Clin. Biochem. 1984; 22:895-904.

In a specific embodiment, antibodies that agonize or antagonize the activity of amyloid beta can be generated. Such antibodies can be tested using the assays described infra for identifying ligands.

One aspect of the invention provides a method of using an antibody against amyloid beta, for example Aβ₁₋₄₂, to diagnose a major depressive disorder in a subject. As amyloid beta levels correlate with the presence of a major depressive episode/disorder in a subject, with the presence of a major episode/depressive disorder being determined by a psychiatric evaluation based on the presence of DSM-IV criteria for Major Depressive Episodes and the presence of significant depressive symptoms as determined by a psychiatric rating scale such as the Hamilton scale, amyloid beta serves as a general biomarker for a major depressive disorder, and may be predictive of the future onset of such disorder. Alternatively, it may also serve as a marker for monitoring efficacy of therapy for such disorder, as described herein. Thus, the antibody compositions and methods provided herein are particularly deemed useful for the diagnosis of a major depressive disorder. In a particular embodiment, the major depressive disorder is the result of build-up of amyloid beta in the brains of individuals suffering from such major depressive disorder. More particularly, the amyloid beta is A β₄₀ and A β₄₂.

The diagnostic method of the invention provides contacting a biological sample such as a biopsy sample, tissue, cell or fluid (e.g., whole blood, plasma, serum, urine, or CSF) isolated from a subject with an antibody which binds amyloid beta (Ghiso et al. FEBS Letters 1997; 408:105-108). The antibody is allowed to bind to the antigen to form an antibody-antigen complex. The conditions and time required to form the antibody-antigen complex may vary and are dependent on the biological sample being tested and the method of detection being used. Once non-specific interactions are removed by, for example, washing the sample, the antibody-antigen complex is detected using any one of the immunoassays described above as well a number of well-known immunoassays used to detect and/or quantitate antigens (see, for example, Harlow and Lane, Antibodies: A Laboratory Manual, Cold Spring Harbor Laboratory, New York (1988) 555-612). Such well-known immunoassays include antibody capture assays, antigen capture assays, and two-antibody sandwich assays. In an antibody capture assay, the antigen is attached to solid support, and labeled antibody is allowed to bind. After washing, the assay is quantitated by measuring the amount of antibody retained on the solid support. In an antigen capture assay, the antibody is attached to a solid support, and labeled antigen is allowed to bind. The unbound proteins are removed by washing, and the assay is quantitated by measuring the amount of antigen that is bound. In a two-antibody sandwich assay, one antibody is bound to a solid support, and the antigen is allowed to bind to this first antibody. The assay is quantitated by measuring the amount of a labeled second antibody that binds to the antigen.

These immunoassays typically rely on labeled antigens, antibodies, or secondary reagents for detection. These proteins may be labeled with radioactive compounds, enzymes, biotin, or fluorochromes. Of these, radioactive labeling may be used for almost all types of assays. Enzyme-conjugated labels are particularly useful when radioactivity must be avoided or when quick results are needed. Biotin-coupled reagents usually are detected with labeled streptavidin. Streptavidin binds tightly and quickly to biotin and may be labeled with radioisotopes or enzymes. Fluorochromes, although requiring expensive equipment for their use, provide a very sensitive method of detection. Those of ordinary skill in the art will know of other suitable labels which may be employed in accordance with the present invention. The binding of these labels to antibodies or fragments thereof may be accomplished using standard techniques such as those described by Kennedy, et al., Clin. Chim. Acta 1976; 70: 1-31), and Schurs, et al. Clin. Chim Acta 1977; 81: 1-40).

In accordance with the diagnostic method of the invention, the presence or absence of the antibody-antigen complex is correlated with the presence or absence in the biological sample of the antigen, or a peptide fragment thereof. A biological sample containing elevated levels of said antigen (amyloid beta) is indicative of a major depressive disorder in a subject from which the biological sample was obtained. Accordingly, the diagnostic method of the invention may be used as part of a routine screen in subjects suspected of having a major depressive disorder or for subjects who may be predisposed to having a major depressive disorder. Moreover, the diagnostic method of the invention may be used alone or in combination with other well-known diagnostic methods to confirm the presence of a major depressive disorder.

The diagnostic method of the invention further provides that an antibody of the invention may be used to monitor the levels of amyloid beta antigen in patient samples at various intervals of drug treatment to identify whether and to which degree the drug treatment is effective in reducing or inhibiting the symptoms associated with such depressive disorder, such reduction being an indication that the therapy may ultimately result in amelioration and/or cure of the disorder. Furthermore, antigen levels may be monitored using an antibody of the invention in studies evaluating efficacy of drug candidates in model systems and in clinical trials. The antigens provide for surrogate biomarkers in biological fluids to non-invasively assess the global status of the major depressive disorder. For example, using an antibody of this invention, antigen levels may be monitored in biological samples of individuals treated with known or unknown therapeutic agents or toxins. This may be accomplished with cell lines in vitro or in model systems and clinical trials, depending on the depressive disorder being investigated. Persistently increased total levels of amyloid beta antigen in biological samples during or immediately after treatment with a drug candidate indicates that the drug candidate has little or no effect on cell proliferation. Likewise, the reduction in total levels of amyloid beta antigen indicates that the drug candidate is effective in reducing or inhibiting the symptoms of the major depressive disorder. Furthermore, the continued reduction of amyloid beta in the subject may ultimately result in full remission of the individual suffering from such depressive disorder. This may provide valuable information at all stages of pre-clinical drug development, clinical drug trials as well as subsequent monitoring of patients undergoing drug treatment.

### Antibody Labels

The amyloid beta proteins of the present invention, antibodies to amyloid beta proteins, and nucleic acids that hybridize to amyloid beta genes (e.g. probes) etc. can all be labeled. Suitable labels include enzymes, fluorophores (e.g., fluorescein isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), and chemiluminescent agents. When a control marker is employed, the same or different labels may be used for the receptor and control marker.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁹Y, ¹²⁵I, ¹³¹I, and ¹⁸⁶Re are used, known currently available counting procedures may be utilized. Such labels may also be appropriate for the nucleic acid probes used in binding studies with amyloid beta. In the instance where the label is an enzyme, detection may be accomplished by any of the presently utilized colorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g. ultraviolet light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 1980; 70:419-439) and in U.S. Pat. No. 4,857,453.

Suitable enzymes include, but are not limited to, alkaline phosphatase and horseradish peroxidase.

In addition, amyloid beta, or a fragment thereof can be modified to contain a marker protein such as green fluorescent protein as described in U.S. Pat. No. 5,625,048 filed Apr. 29, 1997, WO 97/26333, published Jul. 24, 1997 and WO 99/64592 all of which are hereby incorporated by reference in their entireties.

Other labels for use in the invention include magnetic beads or magnetic resonance imaging labels.

In another embodiment, a phosphorylation site can be created on an antibody of the invention for labeling with ³²P, e.g., as described in European Patent No. 0372707 (application No. 89311108.8) by Sidney Pestka, or U.S. Pat. No. 5,459,240, issued Oct. 17, 1995 to Foxwell et al.

As exemplified herein, proteins, including antibodies, can be labeled by metabolic labeling. Metabolic labeling occurs during in vitro incubation of the cells that express the protein in the presence of culture medium supplemented with a metabolic label, such as [³⁵S]-methionine or [³²P]-orthophosphate. In addition to metabolic (or biosynthetic) labeling with [³⁵S]-methionine, the invention further contemplates labeling with [¹⁴C]-amino acids and [³H]-amino acids (with the tritium substituted at non-labile positions).

Quantification of amyloid beta can also be done using staining with Congo red with subsequent image analysis (Kindy, M S et al., Am. J. Pathol. 1998; 152: 1387-1395; Kisilevsky, R. et al., Nat. Med. 1995; 1: 143-148).

### Other Diagnostic Means for Determining Levels of Amyloid Beta

### Cell-Based Reporters and Instrumentation

Cellular screening techniques can be broadly classified into two groups: semi-biochemical approaches that involve the analysis of cell lysates, or live cell assays. Whole cell assay methodologies vary with respect to assay principle, but have largely in common a form of luminescence or fluorescence for detection. Luminescence is a phenomenon in which energy is specifically channeled to a molecule to produce an excited state. Luminescence includes fluorescence, phosphorescence, chemiluminescence and bioluminescence.

An ever-increasing list of fluorescent proteins includes the widely-used GFP derived from Aequorea Victoria and spectral variants thereof. The list includes a variety of fluorescent proteins derived from other marine organisms; bacteria; fungi; algae; dinoflagellates; and certain terrestrial species. These reporters have the advantage of not requiring any exogenous substrates or co-factors for the generation of a signal but do require an external source of radiation for excitation of the intrinsic fluorophore. In addition, the increasing availability of genes encoding a broad spectrum of fluorescent reporter proteins enables the construction of assays tailored for specific applications, cell types, and detection systems.

Different classes of luminescent proteins, luciferases, have been discovered in bacteria and eukaryotes. Luciferases are proteins that catalyze the conversion of a natural substrate into a product that emits light in the visible spectrum and thus require no external radiation source. Monomeric forms of luciferase have been cloned from firefly, Renilla, and other organisms. Firefly luciferase is the most common of the bioluminescent reporters and is a 61 kDa monomeric enzyme that catalyzes a two-step oxidation reaction to yield light. Renilla luciferase is a 31 kDa monomeric enzyme that catalyzes the oxidation of coelenterazine to yield coelenteramide and blue light of 480 nm. Substrates for luciferase are widely available from commercial suppliers such as Promega Corporation and Invitrogen Molecular Probes.

A variety of useful enzymatic reporters are enzymes that either generate a fluorescent signal or are capable of binding small molecules that can be tagged with a fluorescent moiety to serve as a fluorescent probe. For example, dihydrofolate reductase (DHFR) is capable of binding methotrexate with high affinity; a methotrexate-fluorophore conjugate can serve as a quantitative fluorescent reagent for the measurement of the amount of DHFR within a cell. By tagging methotrexate with any of a number of fluorescent molecules such as fluorescein, rhodamine, Texas Red, BODIPY and other commercially available molecules (such as those available from Molecular Probes/Invitrogen and other suppliers) a range variety of fluorescent readouts can be generated. The wide range of techniques of immunohistochemistry and immunocytochemistry can be applied to whole cells. For example, ligands and other probes can be tagged directly with fluorescein or another fluorophore for detection of binding to cellular proteins; or can be tagged with enzymes such as alkaline phosphatase or horseradish peroxidase to enable indirect detection and localization of signal.

Many other enzymes can be used to generate a fluorescent signal in live cells by using specific, cell-permeable substrate that either becomes fluorescent or shifts its fluorescence spectrum upon enzymatic cleavage. For example, substrates for β-lactamase exist whose fluorescence emission properties change in a measurable way upon cleavage of a β-lactam core moiety to which fluorophores are attached. Changes include, shifts in fluorophore absorption or emission wavelengths, or cleavage of a covalent assembly of emmision-absorption-mathched fluorophore pairs that in the covalently-assembled form sustain resonance energy transfer between the two fluorophores that is lost when the two are separated. Membrane-permeant, fluorescent BLA substrates such as the widely-used CCF2/AM allow the measurement of gene expression in live mammalian cells in the absence or presence of compounds from a biologically active chemical library.

Luminescent, fluorescent or bioluminescent signals are easily detected and quantified with any one of a variety of automated and/or high-throughput instrumentation systems including fluorescence multi-well plate readers, fluorescence activated cell sorters (FACS) and automated cell-based imaging systems that provide spatial resolution of the signal. A variety of instrumentation systems have been developed to automate HCS including the automated fluorescence imaging and automated microscopy systems developed by Cellomics, Amersham, TTP, Q3DM, Evotec, Universal Imaging and Zeiss. Fluorescence recovery after photobleaching (FRAP) and time lapse fluorescence microscopy have also been used to study protein mobility in living cells. Although the optical instrumentation and hardware have advanced to the point that any bioluminescent signal can be detected with high sensitivity and high throughput, the existing assay choices are limited either with respect to their range of application, format, biological relevance, or ease of use.

### Transcriptional Reporter Assays

Cell-based reporters are often used to construct transcriptional reporter assays, which allow monitoring of the cellular events associated with signal transduction and gene expression. Reporter gene assays couple the biological activity of a target to the expression of a readily detected enzyme or protein reporter. Based upon the fusion of transcriptional control elements to a variety of reporter genes, these systems “report” the effects of a cascade of signaling events on gene expression inside cells. Synthetic repeats of a particular response element can be inserted upstream of the reporter gene to regulate its expression in response to signaling molecules generated by activation of a specific pathway in a live cell. The variety of transcriptional reporter genes and their application is very broad and includes drug screening systems based on β-galactosidase (β-gal), luciferase, alkaline phosphatase (luminescent assay), GFP, aequorin, and a variety of newer bioluminescent or fluorescent reporters.

In general, transcription reporter assays have the capacity to provide information on the response of a pathway to natural or synthetic chemical agents on one or more biochemical pathways, however they only indirectly measure the effect of an agent on a pathway by measuring the consequence of pathway activation or inhibition, and not the site of action of the compound. For this reason, mammalian cell-based methods have been sought to directly quantitate protein-protein interactions that comprise the functional elements of cellular biochemical pathways and to develop assays for drug discovery based on these pathways.

Cellular Assays for Individual Proteins Tagged with Fluorophores or Luminophores.

Subcellular compartmentalization of signaling proteins is an important phenomenon not only in defining how a biochemical pathway is activated but also in influencing the desired physiological consequence of pathway activation. This aspect of drug discovery has seen a major advance as a result of the cloning and availability of a variety of intrinsically fluorescent proteins with distinct molecular properties.

High-content (also known as high-context) screening (HCS) is a live cell assay approach that relies upon image-based analysis of cells to detect the subcellular location and redistribution of proteins in response to stimuli or inhibitors of cellular processes. Fluorescent probes can be used in HCS; for example, receptor internalization can be measured using a fluorescently-labeled ligand that binds to the transferrin receptor. Often, individual proteins are either expressed as fusion proteins, where the protein of interest is fused to a detectable moiety such as GFP, or are detected by immunocytochemistry after fixation, such as by the use of an antibody conjugated to Cy3 or another suitable dye. In this way, the subcellular location of a protein can be imaged and tracked in real time. One of the largest areas of development is in applications of GFP color-shifted mutants and other more recently isolated new fluorescent proteins, which allow the development of increasingly advanced live cell assays such as multi-color assays. A range of GFP assays have been developed to analyze key intracellular signaling pathways by following the redistribution of GFP fusion proteins in live cells. For drug screening by HCS the objective is to identify therapeutic compounds that block disease pathways by inhibiting the movement of key signaling proteins to their site of action within the cell.

Tagging a protein with a fluorophore or a luminophore enables tracking of that particular protein in response to cell stimuli or inhibitors. For example, the activation of cell signaling by TNF can be detected by expressing the p65 subunit of the NFkB transcription complex as a GFP fusion and then following the redistribution of fluorescence from the cytosolic compartment to the nuclear compartment of the cell within minutes after TNF stimulation of live cells (J A Schmid et al., J. Biol. Chem. 2000; 275: 17035-17042). What has been unique about these approaches is the ability to allow monitoring of the dynamics of individual protein movements in living cells, thus addressing both the spatial and temporal aspects of signaling.

Methods of Treating a Mood Disorder such as a Major or Minor Depressive Disorder or Dysthymia

Another aspect of the invention provides for methods of treating and/or preventing a mood disorder such as a major or minor depressive disorder or dysthymia comprising administering a therapeutically effective amount of an agent that reduces brain amyloid beta levels. Such reduction of brain amyloid beta levels may be the result of treatment with an agent which either prevents the production or generation of amyloid beta (such production or generation occurring through the cleavage of beta amyloid precursor protein or APP), or which prevents the aggregation of amyloid beta fibrils and deposition in cerebral parenchymal tissues and blood vessels, or by preventing the formation of the oligomeric forms of A β, A β₁₋₄₀ or A β₁₋₄₂, or which increases the degradation of amyloid beta, or which increases the clearance of amyloid beta from the brain, or which facilitates the peripheral metabolism and clearance of amyloid beta. In another aspect the method of treating or preventing a mood disorder such as a major or minor depressive disorder or dysthymia comprises administration of an agent that might prevent or interfere with A β-induced neurotoxicity. In a particular embodiment, the amyloid beta is amyloid β₁₋₄₀ or amyloid β₁₋₄₂ or fragments thereof. In another embodiment of the present invention, it is envisioned that circulating levels of amyloid beta, including A β₄₀ or A β₄₂ and oligomeric forms may be damaging (eg. neurotoxic) even if not deposited. That is, the present invention contemplates that soluble, non-fibrillary forms of A β, in particular, A β₁₋₄₂ may be neurotoxic and can lead to neuronal damage or alterations in neurotransmitter levels or perturbations in neurotransmission through interference with signaling systems, the result of which is a Major Depressive Episode or Disorder. Accordingly, the levels of circulating A β as found in plasma or whole blood may be diagnostic of a Major Depressive Disorder or Episode or predictive of a risk for developing such disorders. The studies shown herein have demonstrated that elevated A β₄₂ can be found in the plasma of both early onset depressive patients (before the age of 60) as well as late onset depressive patients (after the age of 60), thus suggesting that elevated plasma A β₄₂ levels may play a role in depressive symptoms and depressive episodes/disorders in the young as well as the elderly population. Thus, treatment strategies contemplated for the elderly with respect to the lowering of A β, or for interfering with A β-mediated neurotoxicity, especially A β₁₋₄₂, are also contemplated for the younger population wherein higher than normal levels of A β are associated with a Major Depressive Episode/Disorder.

Another aspect of the invention provides for combination therapy with an agent that reduces brain amyloid beta and use of at least one other (a second) therapeutic agent. In a preferred embodiment, a composition comprising an agent/compound that reduces brain amyloid beta is administered concurrently with the administration of a second therapeutic agent, which can be part of the same composition as or in a different composition from that comprising the agent/compound that reduces brain amyloid beta. In another embodiment, a composition comprising a compound that reduces brain amyloid beta is administered prior or subsequent to administration of a second therapeutic agent. As many of the disorders for which the compounds of the invention are useful in treating are chronic, in one embodiment combination therapy involves alternating between administering a composition comprising a compound that reduces brain amyloid beta and a composition comprising a second therapeutic agent, e.g., to minimize the toxicity associated with a particular drug. The duration of administration of the agent/compound that reduces brain amyloid beta or and/or second therapeutic agent can be, e.g., one month, three months, six months, a year, or for more extended periods, or on an alternate or intermittent schedule. In certain embodiments, when a compound that reduces brain amyloid beta is administered concurrently with a second therapeutic agent that potentially produces adverse side effects including, but not limited to, toxicity, the second therapeutic agent can advantageously be administered at a dose that falls below the threshold at which the adverse side is elicited. In another particular embodiment, the second drug that is used is effective at treating a mood disorder such as a major or minor depressive disorder or dysthymia. In yet another particular embodiment, the second drug is administered concurrently, prior to, or after the agent that reduces brain amyloid beta levels. In yet another particular embodiment, the second drug is selected from the group consisting of Selective Serotonin Reuptake Inhibitors (SSRI), tricyclic antidepressants, and monoamine oxidase (MAO) inhibitors. In a more particular embodiment, the Selective Serotonin Reuptake Inhibitor is selected from the group consisting of citalopram, escitalopram HBr, fluvoxamine, paroxetine, fluoxetine, and sertraline. In a more particular embodiment, the tricyclic antidepressant is selected from the group consisting of amitriptyline, desipramine, doxepin, protriptyline, trimipramine and nortriptyline. In a more particular embodiment, the monoamine oxidase inhibitor is selected from the group consisting of phenelzine and tranylcypromine. In a more particular embodiment the second drug is selected from the group consisting of drugs that act as inhibitors of neuronal reuptake of both serotonin (5-HT) and norepinephrine (NE). In a more particular embodiment, the inhibitor of neuronal reuptake of both serotonin and norepinephrine is venlafaxine and duloxetine.

In another particular embodiment, the method further comprises treating with an agent that may interfere with the actions of amyloid beta on neurons and other brain cells that may contribute to the pathophysiology of a mood disorder such as a major or minor depressive disorder or dysthymia in humans.

While it is possible for the agent to be administered alone, it is preferable to present it as a pharmaceutical composition. A generally recognized compendium of methods and ingredients of pharmaceutical compositions is Remington's Pharmaceutical Science, 15th ed., Mack Publishing Company, Easton, Pa. The active ingredient may comprise, for topical administration, from 0.001% to 10% w/w, e.g., from 1% to 2% by weight of the composition, although it may comprise as much as 10% w/w but preferably not in excess of 5% w/w and more preferably from 0.1% to 1% w/w of the composition.

Amyloid beta formation may be retarded, or the progression of amyloidosis may be prevented by an agent of the present invention, including but not limited to a synthetic organic molecule, a protein or peptide, an enzyme, a carbohydrate, a nucleic acid, an antisense oligonucleotide, a small or short interfering RNA molecule (siRNA), or an antibody or fragment thereof. In one particular embodiment, the agent can be used to treat a mood disorder such as a major or minor depressive disorder or dysthymia. In another particular embodiment, and as noted above, the effects of the agent are through its ability to prevent production or generation of amyloid beta from beta amyloid precursor protein (APP), or by increasing the degradation of amyloid beta, or by increasing the clearance of amyloid beta from the brain, or by facilitating the peripheral metabolism and clearance of amyloid beta.

For example, amyloid beta formation could be inhibited by peptides homologous to A β (position 17-21) which have a similar degree of hydrophobicity, but also have a very low propensity to adopt a β-sheet conformation due to the presence of proline residues (anti-β-sheet peptides or β-sheet inhibitors) (Soto, et al., (1996), Biophys Res Commun., 1996; 226(3):672-80). An 11-residue peptide with these characteristics binds to A β, inhibits A β fibril formation and partially disaggregates preformed fibrils in vitro. Shorter anti-β-sheet peptides and analogs containing D-amino acids are also able to inhibit A β fibrillogenesis. The latter are more resistant to proteolytic degradation and may serve as a starting point to design more efficient peptides derivatives to inhibit amyloidogenesis in vivo.

Alternatively, it has been determined that N-methyl amino acid containing congeners of the hydrophobic “core domain” of A β inhibit the fibrillogenesis of full-length A β. These peptides also disassemble preformed fibrils of full-length A β. A key feature of the inhibitor peptides is that they contain N-methyl amino acids in alternating positions of the sequence. The most potent of these inhibitors, termed A β 16-22m, has the sequence NH(2)-K(Me-L)V(Me-F)F(Me-A)E-CONH(2) (SEQ ID NO: 6). These inhibitors appear to act by binding to growth sites of A β nuclei and/or fibrils and preventing the propagation of the network of hydrogen bonds that is essential for the formation of an extended β-sheet fibril. (Gordon, et al., Biochemistry. 2001; July 27; 40(28): 8237-45). Furthermore, it has been shown that single N-methyl amino acid-containing peptides related to the central hydrophobic region β₁₆₋₂₀ (Lys-Leu-Val-Phe-Phe (SEQ ID NO: 7) of the β-amyloid protein are able to reduce the cytotoxicity of natural β₁₋₄₂ in PC12 cell cultures. N-methyl phenylalanine analogs yield statistically significant increments in cell viability (Student's t-test<0.01%) and are nontoxic in the same assay (M. Cruz, et al., The Journal of Peptide Research, 2004; 63(3): 324).

In addition, it has been shown that phenserine, a third generation acetylcholinesterase inhibitor (AChE-inhibitor), has the ability to reduce both β amyloid precursor protein (APP) and β amyloid peptide (amyloid-β) formation in the brain.

Other investigators have reported that injection of animals with low molecular weight heparins (enoxaprin and dalteparin) demonstrated a reduction in amyloid beta deposition. Moreover, these compounds were capable of arresting the progression of amyloid beta deposits and amyloid beta peptide fibril formation by impeding the structural changes necessary for fibril formation. Not only was amyloid beta progression retarded, but these molecules enhanced the clearance of established amyloid beta fibrils (Zhu, H. et al., Mol. Med. 2001; 7(8):517-522).

Furthermore, it has been shown that monoclonal antibodies are capable of inhibiting in vitro fibrillar aggregation of the Alzheimer β-amyloid peptide (Solomon B, et al., Proc Natl Acad Sci USA, 1996; 93(1):452-5). In one study, two monoclonal antibodies (mAbs), AMY-33 and 6F/3D, were raised against β-amyloid fragments spanning amino acid residues 1-28 and 8-17 of the β-amyloid peptide chain, respectively. In vitro aggregation of β-amyloid peptide was induced by incubation for 3 hours at 37° C. and monitored by ELISA, negative staining electron microscopy, and fluorimetric studies. The investigators found that the monoclonal antibodies prevented the aggregation of β-amyloid peptide. Moreover, the inhibitory effect appeared to be related to the localization of the antibody-binding sites and the nature of the aggregating agents (Solomon B, et al., Proc Natl Acad Sci USA, 1996; 93(1):452-5).

It may also be possible to prevent the effects of amyloid beta by administering an agent that effects a regulatory element necessary for its expression. Alternatively, the effects of amyloid beta in a mood disorder such as a major or minor depressive disorder or dysthymia may be addressed by taking into account the effects of A β in the inflammatory process. For example, studies by Suo et al. suggest that β-amyloid (A β)-induced inflammatory reactions may partially drive the pathogenesis of Alzheimer's disease (AD) and in cerebral amyloid angiopathy (CAA). To evaluate the roles of A β in the inflammatory processes in vascular tissues, they tested the ability of A β to trigger inflammatory responses in cultured human vascular cells. They found that stimulation with A β dose-dependently increased the expression of CD40, and secretion of interferon-gamma (IFN-gamma) and interleukin-1 β (IL-1 β) in endothelial cells. A β also induced expression of IFN-gamma receptor (IFN-gammaR) both in endothelial and smooth muscle cells. Characterization of the A β-induced inflammatory responses in the vascular cells showed that the ligation of CD40 further increased cytokine production and/or the expression of IFN-gammaR. Moreover, IL-1 β and IFN-gamma synergistically increased the A β-induced expression of CD40 and IFN-gammaR. They also found that A β induced expression of adhesion molecules, and that cytokine production and interaction of CD40-CD40 ligand (CD40L) further increased the A β-induced expression of adhesion molecules in these same cells. These results suggest that A β can function as an inflammatory stimulator to activate vascular cells and induces an auto-amplified inflammatory molecular cascade, through interactions among adhesion molecules, CD40-CD40L and cytokines. Additionally, A β1-42, the more pathologic form of A β, induces much stronger effects in endothelial cells than in smooth muscle cells, while the reverse is true for A β1-40. Collectively, these findings support the hypothesis that the A β-induced inflammatory responses in vascular cells may play a significant role in the pathogenesis of CAA and AD (Suo et al., Brain Res 1998; 807(1-2): 110-7).

Studies have also focused on a means of enhancing the degradation and clearance of A β. Studies by Qiu et al. have shown increased degradation of amyloid β protein by a metalloprotease secreted by microglia. Such a metalloprotease was identified in a microglial cell line, BV-2 (Qiu, et al., J. Biol. Chem. 1997; 272(10): 6641-6). Substrate analysis revealed that the enzyme responsible for the degradation of A β₁₋₄₀ and A β₁₋₄₂ was a non-matrix metalloprotease. Thus, it may be possible to develop novel therapeutics that act to enhance the activity of this enzyme or to identify other enzymes that act in a similar manner to degrade the amyloid beta proteins, or alternatively, to develop a means of directing specific enzymes with such amyloid beta degrading activity to the site where needed.

Another enzyme, neprilysin, has been shown to be a major A β degrading enzyme in vivo. Studies by Hama, et al. investigated whether or not manipulation of neprilysin activity in the brain would be an effective strategy for regulating A β levels. Neprilysin was expressed in primary cortical neurons using a Sindbis viral vector and the effect on A β metabolism was examined. The investigators showed that the corresponding recombinant protein, expressed in the cell bodies and processes, exhibited thiorphan-sensitive endopeptidase activity, whereas a mutant neprilysin with an amino acid substitution in the active site did not show any such activity. Expression of the wild-type neprilysin, but not the mutant, resulted in a dose-dependent decrease in both the A β 40 and 42 levels in the culture media (Hama, et al., Journal of Biochemistry, 2001; 130(6): 721-726). Moreover, neprilysin expression also resulted in reducing cell-associated A β, which could be more neurotoxic than extracellular A β. These results indicate that the manipulation of neprilysin activity in neurons, the major source of A β in the brain, would be a relevant strategy for controlling the A β levels and thus the A β-associated pathology in brain tissues. In the matter of the present invention, such a strategy could be used to treat a mood disorder such as a major or minor depressive disorder or dysthymia associated with elevated levels of A β peptides.

Studies have also shown that endothelin converting enzyme (ECE) has been shown to play a fundamental role in amyloid metabolic pathways. A published study (Journal of Biological Chemistry: 2001; 276(27): 24540-8,) reported that both ECE-1a and ECE-1b activation reduced the intracellular generation of A β 40 and A β 42, and that ECE-1a may also be able to degrade preexisting protein. The development of strategies able to stimulate or over-express ECE could offer a novel therapy of treating a major depressive disorder characterized by elevated levels of A β 40 and A β 42.

Furthermore, it has been shown that a modest increase in astroglial TGF-β 1 production in aged transgenic mice expressing the human β-amyloid precursor protein (hAPP) results in a three-fold reduction in the number of parenchymal amyloid plaques, a 50% reduction in the overall A β load in the hippocampus and neocortex, and a decrease in the number of dystrophic neuritis (Wyss-Coray, et al., Nat Med 2001; 7(5): 612-8). These investigators found that in mice expressing hAPP and TGF-β 1, A β accumulated substantially in cerebral blood vessels, but not in parenchymal plaques. Furthermore, in human cases of AD, A β immunoreactivity associated with parenchymal plaques was inversely correlated with A β in blood vessels and cortical TGF-β 1 mRNA levels. The reduction of parenchymal plaques in hAPP/TGF-β 1 mice was associated with a strong activation of microglia and an increase in inflammatory mediators. Recombinant TGF-β 1 stimulated A β clearance in microglial cell cultures. These results demonstrate that TGF-β 1 is an important modifier of amyloid beta deposition in vivo and indicate that TGF-β 1 might promote microglial processes that inhibit the accumulation of A β in the brain parenchyma. Accordingly, agents that modulate TGF-β activity may prove useful to treat major depressive disorders by virtue of their effect on A β deposition and/or clearance.

In studies related to the role of amyloid beta in Alzheimer's disease, several groups are exploring the possibility that A β-based vaccines can help the brain in Alzheimer's patients by increasing its clearance. Work by Lemere et al. demonstrated that vaccinating monkeys with synthetic amyloid beta peptide enhanced the clearance of amyloid beta protein from the brain and into the blood. In this study, five aged Caribbean vervet monkeys were given eight injections of the amyloid beta peptide over a period of nine-months. Five control monkeys did not receive the treatment. The investigators found that all of the immunized monkeys made antibodies to amyloid beta peptide. These antibodies were found in blood and, in lower amounts, in cerebrospinal fluid (CSF). More importantly, the investigators found that between 22 and 42 days after immunization, levels of amyloid beta protein dropped in the CSF and increased in the blood and no plaques were found in the immunized animals. These findings suggest that the antibodies bound to amyloid beta protein and increased its slow release from the CSF to the blood for clearance (http://www.ahaforg/whatsnew/AD_Hope_Nov_{—}2003.htm). One possible drawback to this approach is the finding that foreign β-amyloid could have unwanted consequences, such as contributing to the death of brain cells or triggering harmful immune system responses. One possible concern is the induction of an inflammatory response in the brain. Thus, it may be necessary to administer other anti-inflammatory compounds at the time of vaccination. The present invention provides for such combination therapies.

One alternative is to find nontoxic molecules that will boost the clearance of β-amyloid without inducing an inflammatory response. Agadjanyan et al. have found several molecules that mimic β-amyloid (termed “mimotopes”) in mice, and they are now looking for corresponding mimotopes in humans. Future studies by this group will concentrate on identification of β-amyloid mimotopes in the blood from patients with Alzheimer's disease. These molecules will then be tested in a mouse model of Alzheimer's disease to determine whether vaccination with human mimotopes can reduce mouse brain levels of β-amyloid and reverse memory deficits, without side effects.

Any of the above agents, whether they are small organic molecules, enzymes, peptides or antibodies, which prevent the production or generation of A β through cleavage of APP, or aggregation of amyloid beta fibrils, or promote the degradation and/or clearance of amyloid beta peptides, may be useful for treatment of a major depressive disorder. Such agents may be used alone, or in combination with other therapies such as anti-inflammatories or pain medications or other standard treatments for depression, such as those noted herein. These other therapies may be administered concurrently, or may be given prior to, or after, the agents described above.

### Pharmaceutical Compositions

Another aspect of the invention provides for pharmaceutical compositions for the treatment of a mood disorder such as a major or minor depressive disorder or dysthymia. The pharmaceutical composition comprises an agent that prevents the production or generation of amyloid beta through cleavage of APP, such as an inhibitor of gamma or beta secretase, and a pharmaceutically acceptable carrier, or an agent that prevents aggregation of amyloid beta fibrils and deposition in cerebral parenchymal tissues and blood vessels, or by preventing the formation of oligomeric forms of A β, A β₁₋₄₀ or A β₁₋₄₂, or an agent that increases the degradation of amyloid beta and a pharmaceutically acceptable carrier, or an agent that increases the clearance of amyloid beta from the brain and a pharmaceutically acceptable carrier, or an agent that facilitates the metabolism of amyloid beta and a pharmaceutically acceptable carrier. The pharmaceutical compositions may be formulated for oral delivery or parenteral delivery. Furthermore, the pharmaceutical compositions are formulated for delivery by a route selected from the group consisting of intravenous, intramuscular, oral, subcutaneous, intrathecal, intracranial and intraventricular. In a particular embodiment, the amyloid beta is amyloid β₁₋₄₀ or amyloid β₁₋₄₂.

The compositions for parenteral administration will commonly comprise a solution of any of the agents described above, including an antibody or fragment thereof or a protein or small organic molecule of the invention or a cocktail thereof dissolved in an acceptable carrier, preferably an aqueous carrier. A variety of aqueous carriers may be employed, e.g., water, buffered water, 0.4% saline, 0.3% glycine, and the like. These solutions are sterile and generally free of particulate matter. These solutions may be sterilized by conventional, well-known sterilization techniques. The compositions may contain pharmaceutically acceptable auxiliary substances as required to approximate physiological conditions such as pH adjusting and buffering agents, etc. The concentration of the agent of the invention in such pharmaceutical compositions may vary widely, i.e., from less than about 0.5%, usually at or at least about 1% to as much as 15 or 20% by weight, and will be selected primarily based on fluid volumes, viscosities, etc., according to the particular mode of administration selected.

Thus, a pharmaceutical composition of the invention for intramuscular injection could be prepared to contain 1 ml sterile buffered water, and 50 mg of agent of the invention. Similarly, a pharmaceutical composition of the invention for intravenous infusion could be made up to contain 250 ml of sterile Ringer's solution, and 150 mg of the agent of the invention. Actual methods for preparing parenterally administrable compositions are well-known or will be apparent to those skilled in the art, and are described in more detail in, e.g., Remington's Pharmaceutical Science, 15th ed., Mack Publishing Company, Easton, Pa.

The agent of the invention may be lyophilized for storage and reconstituted in a suitable carrier prior to use.

The pharmaceutical composition of the invention may be administered for prophylactic and/or therapeutic treatments. In therapeutic application, compositions are administered to a subject already suffering from a mood disorder such as a major or minor depressive disorder or dysthymia, in an amount sufficient to cure or at least partially arrest the disorder and its complications. In prophylactic applications, compositions containing the present agents are administered to a subject not already in a disease state but one that may be predisposed to a depressive disorder to enhance the subject's resistance to such disorder.

It will be recognized by one of skill in the art that the optimal quantity and spacing of individual dosages of an agent of the invention will be determined by the nature and extent of the depressive disorder being treated, the form, route and site of administration, and the particular animal being treated, and that such optimums may be determined by conventional techniques. It will also be appreciated by one of skill in the art that the optimal course of treatment, i.e., the number of doses of an agent of the invention given per day for a defined number of days, may be ascertained by those skilled in the art using conventional course of treatment determination tests.

### Administration of the Pharmaceutical Compositions

According to the present invention, a therapeutic composition, e.g., an inhibitor of amyloid beta production or generation through cleavage of amyloid precursor protein (APP), an inhibitor of amyloid beta aggregation into fibrils, an enhancer of amyloid beta clearance from the brain, an enhancer of amyloid beta degradation, or an agent that interferes with the neurotoxic effects of amyloid beta, and a pharmaceutically acceptable carrier of the invention or an agent such as a small organic molecule or an antibody that performs one of these functions, may be introduced orally or parenterally, e.g. intramuscularly, intravenously, subcutaneously, transmucosally, or nasally. Alternatively, administration is by intracranial, intrathecal or intraventricular administration. In addition, the therapeutic composition can be placed (e.g., injected) into the bloodstream after coupling the agent to a carrier that will allow the agent-carrier complex to cross the blood-brain barrier.

In a preferred aspect, the agent of the present invention can cross cellular or nuclear membranes, which would allow for intravenous or oral administration. Strategies are available for such crossing, including but not limited to, increasing the hydrophobic nature of a molecule; introducing the molecule as a conjugate to a carrier, such as a ligand to a specific receptor, targeted to a receptor; and the like.

The present invention also provides for conjugating targeting molecules to the agent of the invention. “Targeting molecule” as used herein shall mean a molecule which, when administered in vivo, localizes to desired location(s). In various embodiments, the targeting molecule can be a peptide or protein, antibody, lectin, carbohydrate, or steroid. In one embodiment, the targeting molecule is a peptide ligand of a receptor on the target cell. In a specific embodiment, the targeting molecule is an antibody. Preferably, the targeting molecule is a monoclonal antibody. In one embodiment, to facilitate crosslinking, the antibody can be reduced to two heavy and light chain heterodimers, or the F(ab′)₂ fragment can be reduced, and crosslinked to the agent via a reduced sulfhydryl.

Antibodies for use as targeting molecule are specific for a cell surface antigen. In one embodiment, the antigen is a receptor. For example, an antibody specific for a receptor on a neuronal cell can be used. This invention further provides for the use of other targeting molecules, such as lectins, carbohydrates, proteins and steroids.

In another embodiment, the therapeutic compound can be delivered in a vesicle, in particular a liposome (see Langer, Science, 1990; 249: 1527-1533; Treat et al., (1989) in Liposomes in the Therapy of Infectious Disease and Cancer, Lopez-Berestein and Fidler (eds.), Liss: New York, pp. 353-365; Lopez-Berestein, ibid., pp. 317-327; see generally ibid.).

In yet another embodiment, the therapeutic compound can be delivered in a controlled release system. For example, the agent may be administered using intravenous infusion, an implantable osmotic pump, a transdermal patch, liposomes, or other modes of administration. In one embodiment, a pump may be used (see Langer (1990) supra; Sefton, CRC Crit. Ref. Biomed. Eng., 1987; 14: 201; Buchwald et al., Surgery, 1989; 88: 507; Saudek et al., N Engl. J. Med., 1989; 321: 574). In another embodiment, polymeric materials can be used (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger et al., J. Macromol. Sci. Rev. Macromol. Chem., 1983; 23: 61; see also Levy et al., Science, 1985; 228: 190; During et al., Ann. Neurol., 1989; 25: 351; Howard et al., J. Neurosurg., 1989; 71: 105). In yet another embodiment, a controlled release system can be placed in proximity of the therapeutic target, i.e., the brain, thus requiring only a fraction of the systemic dose (see, e.g., Goodson, in Medical Applications of Controlled Release, supra, 1984; 2: 115-138).

### Screening Assays for Identification of Novel Agents for Treating a Mood Disorder Such as a Major or Minor Depressive Disorder or Dysthymia

A still further aspect of the invention relates to screening assays to identify agents which inhibit the production or generation of A β by cleavage of APP or the aggregation of amyloid beta fibrils or to enhance the degradation or clearance of A β. The invention encompasses both in vivo and in vitro assays to screen small molecules, compounds, recombinant proteins, peptides, enzymes, nucleic acids, antibodies, etc. which exhibit any of the above activities.

In a preferred embodiment, the binding of the agent is determined through the use of competitive binding assays. The competitor is an antibody of the invention known to bind to amyloid beta protein, including A β₄₀ and A β₄₂, or fragments thereof. Competitive screening assays may be done by combining the amyloid beta protein and an antibody of the invention in a first sample. A second sample comprises a test agent, amyloid beta and an antibody of the invention. The binding of the antibody is determined for both samples, and a change, or difference in binding between the two samples indicates the presence of a test agent capable of binding to amyloid beta and potentially modulating its conformational structure and/or activity. That is, if the binding of the antibody is different in the second sample relative to the first sample, the test agent is capable of binding to amyloid beta protein. Similar designs that utilize antibodies of this invention for the identification of non-antibody compounds that bind to amyloid beta are obvious to those skilled in the art.

One variation provides that the agent is labeled. Either the agent, or the competitor, or both, is added first to amyloid beta protein for a time sufficient to allow binding. Incubations may be performed at any temperature which facilitates optimal activity, typically between 4° C. and 40° C. Incubation periods are selected for optimum activity, but may also be optimized to facilitate rapid high-throughput screening. Typically between 0.1 and 1 hour will be sufficient. Excess reagent is generally removed or washed away. The second component is then added, and the presence or absence of the labeled component is followed, to indicate binding.

It is preferred that the competitor is added first, followed by the test agent. Displacement of the competing antibody of this invention is an indication that the test agent is binding to amyloid beta protein and thus is capable of binding to, and potentially modulating, the conformational structure and/or activity of amyloid beta protein. In this reaction either component may be labeled. Thus, for example, if the competitor is labeled, the presence of label in the wash solution indicates displacement by the agent. Alternatively, if the test agent is labeled, the presence of the label on the support indicates displacement.

Alternatively, the test agent is added first, with incubation and washing, followed by the competitor. The absence of binding by the competitor may indicate the test agent is bound to amyloid beta protein with a higher affinity. Thus, if the agent is labeled, the presence of the label on the support, coupled with a lack of competitor binding, may indicate the test agent is capable of binding to amyloid beta protein.

The agents encompass numerous chemical classes, though typically they are organic molecules, preferably small organic compounds having a molecular weight of more than 100 and less than about 3,500 daltons. Agents comprise functional groups necessary for structural interaction with proteins, particularly hydrogen bonding, and typically include at least an amine, carbonyl, hydroxyl or carboxyl group, preferably at least two of the functional chemical groups. The agents often comprise cyclical carbon or heterocyclic structures and/or aromatic or polyaromatic structures substituted with one or more of the above functional groups. Agents may also be found among biomolecules including peptides, saccharides, fatty acids, steroids, purines, pyrimidines, derivatives, structural analogs or combinations thereof.

Agents are obtained from a wide variety of sources including libraries of synthetic or natural compounds.

Alternatively, the antibodies of this invention may be used for the design and synthesis of either peptide or non-peptide compounds (mimetics) that specifically bind to amyloid beta.

The assays provided use amyloid beta protein, A β₄₀, A β₄₂, or fragments of any of these molecules. In addition, the assays described herein may use either isolated amyloid beta, A β₄₀, A β₄₂, or fragments thereof or cells expressing these molecules or animal models that express these molecules.

A variety of other reagents may be included in the screening assays. These include reagents like salts, neutral proteins, e.g. albumin, detergents, etc. which may be used to facilitate optimal protein-protein binding and/or reduce non-specific or background interactions. Also, reagents that otherwise improve the efficiency of the assay, such as protease inhibitors, nuclease inhibitors, anti-microbial agents, and the like may be used. The mixture of components may be added in any order that provides for the requisite binding.

The methods of the invention are used to identify compounds that inhibit the production or generation of A β through cleavage of APP, such as a gamma or beta secretase inhibitor, or the aggregation of amyloid beta fibrils, or which enhance the degradation or clearance of A β, and are therefore useful in the treatment of disorders, diseases or conditions attributed to elevated levels of amyloid beta, in particular, A β₄₂ or fragments thereof. In the present invention, such elevated levels of amyloid beta have been found in the plasma of patients suffering from a major depressive disorder and have been attributed to development and/or progression of such disorders. A depressed mood is a common feature not only in Major Depressive Episodes/Disorders, but in other mood disorders such as Dysthymic Disorder or Bipolar Disorder. Accordingly, major depressive disorders which can be treated by the methods and compositions provided herein include, but are not limited to, Major Depressive Episodes and/or Disorders. To the extent that other mood disorders such as Dysthymic Disorder, bipolar disorders, and the like, may share abnormal or elevated A β levels, in particular A β₁₋₄₂ such as has been observed in Major Depressive Episodes/Disorders, then therapies that are applicable to Major Depressive Episodes/Disorders as related to the reduction in A β may also be applicable to treatment of these disorders. Since an increase in A β may interfere with neurotransmitters or with their corresponding signaling systems, which may play a role in other disorders such as anxiety or Schizophrenia, then therapies as defined herein to lower levels of A β peptides may be useful for the treatment of these disorders, or the depression associated with these disorders, as well. Furthermore, the use of agents as described herein to treat depressive episodes or disorders may be contemplated as stand-alone therapy or may be used as adjunct therapy with other standard forms of therapy or treatment regimens for depression. It is not yet clear that elevated plasma A β levels, eg. Aβ₄₂, are related to elevations in brain Aβ₄₂ levels in humans. However, there are a number of preclinical studies, which provide strong evidence that brain and plasma levels are in a dynamic equilibrium (Bading, et al. Drug Target, 2002, 10: 359-368; Poduslo, et al., Neurobiol Dis. 1999; 6: 190-199; Zlokovic, et al., Biochem Biophys Res Commun. 1994; 205: 1431-1437; Tanzi, et al., Neuron. 2004; 43: 605-608). This may suggest that a treatment that reduces peripheral levels of A β, eg. Aβ₄₂, might also lower the brain levels of A β, eg. Aβ₄₂, as well.

In the same manner that a small organic molecule or enzyme or mimotope having the desired activity and characteristics noted above, e.g. a molecule that blocks the production or generation of A β, or blocks the aggregation of amyloid beta, or enhances its degradation or clearance, may be administered to treat a mood disorder such as a major or minor depressive disorder or dysthymia, so may an antibody specific for amyloid beta, A β₄₀, A β₄₂ or fragments thereof be administered to treat a mood disorder such as a major or minor depressive disorder or dysthymia. Accordingly, a further aspect of the invention provides methods for treating a mood disorder such as a major or minor depressive disorder or dysthymia by administering to a subject with such disorder an antibody which binds to and therefore blocks the functionally significant region(s) of amyloid beta, A β₄₀ and A β₄₂ or fragments thereof. As one of skill in the art may appreciate, the pharmaceutical compositions comprising the antibody and a pharmaceutically acceptable carrier, as well as the route of administration of such pharmaceutical compositions, would be similar to those provided above for the other agents of this invention. The optimal quantity and spacing of individual dosages of an agent of the invention will be determined by the nature of the agent, the nature and extent of the disorder being treated, the form, route and site of administration, and the particular animal being treated. Such optimums may be determined by conventional techniques of monitoring clinical depression.

### EXAMPLES

The following examples are put forth so as to provide those of ordinary skill in the art with a complete disclosure and description of how to assess the levels of amyloid beta in a population of clinically depressed patients, and are not intended to limit the scope of what the inventors regard as their invention.

### Example 1

### Methods

### Subjects

Out of a total sample of 131 healthy elderly subjects, 51 agreed to a lumbar puncture. Out of these, analysis was restricted to 47 individuals, who had a high level of cognitive function (MMSE score of 28 or higher) and no gross white matter pathology as determined through MRI.

TABLE 1
Demographics

| | Controls | Depressed | |
|---|---|---|---|
| | N = 19 | N = 28 | T-test or X² |
| Age, in years | 68.1 (7.3) | 66.5 (5.4) | t(45) = .835, p = .408 |
| Education, in years | 16.7 (2.7) | 16.5 (2.7) | t(44) = .274, p = .785 a |
| BMI | 28.1 (4.7) | 28.8 (6.7) | t(45) = .378, p = .707 |
| HAM-D | 1.2 (1.9) | 14.9 (8.8) | t(45) = 8.02, p < .001 ** |
| MMSE | 29.5 (.5) | 29.8 (.6) | t(45) = 1.56, p = .126 |
| Diabetes, % (n) | 21% (4) | 18% (5) | X²(1) = .08, p = .785 |
| Female, % (n) | 63% (12) | 36% (10) | X²(1) = 2.410, p = .121 |
| APOE4, % (n) | 26% (5) | 39% (11) | X²(1) = .369, p = .544 |

Note.
Values are means (standard deviations) unless otherwise indicated.
BMI = Body-mass index;
HAM-D = Hamilton Depression Scale;
MMSE = Mini-mental State Exam score;
a = one subject's years of education was not available;
* = significant at the .05 level or below;
** = significant at the .01 level or below.

The study was conducted over 4 visits, generally each 1 week apart. On visit 1, medical and psychiatric history and vital signs were obtained. Participants also underwent a psychiatric evaluation and were classified as either MDD or normal using the SCID for DSM IV; global cognitive status was assessed using the MMSE and the Clinical Dementia Rating. At visit 2, participants received an MRI scan of the head to quantify the magnitude of vascular brain pathology. At visit 3, subjects underwent a comprehensive neuropsychological assessment. On visit 4 a lumbar puncture was performed by a neuroradiologist under guided fluoroscopy.

CSF A β and tau markers were determined. CSF A β was determined by electrochemiluminescence technology, using the MS6000 Human A β Ultra-Sensitive Kit. CSF isoprostanes were determined at Temple University.

### Analysis

First, a series oft-tests and χ² tests were conducted to ascertain that subjects in the MDD and control groups did not differ over dimensions not associated with depression (i.e., MMSE, years of education, BMI, age, incidence of diabetes, gender, and APOE status). Second, we conducted a series oft-tests comparing subjects with MDD and controls over all remaining CSF variables.

### Results

Aside from MDD status, the subjects did not differ in age, education, or presence of the APOE ε4 allele, which is strongly associated with AD. As expected, subjects with MDD had a significantly higher score on the Hamilton Depression scale (HAM-D, 17-item scale; p<0.001).

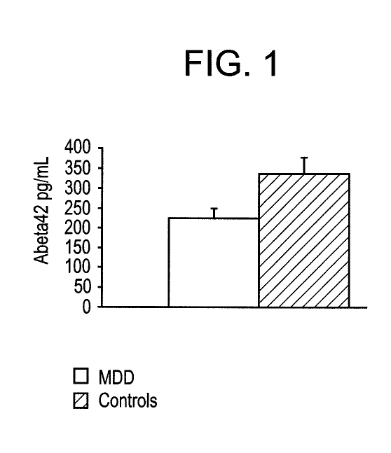

A β 42 [t(45)=2.471, p=0.017] differed significantly across conditions (see FIG. 1), whereas A β 40 [p=0.072], T [p=0.162] and P tau [p=0.712] did not reach significance. The difference in A β 42 was still present [t(29)=2.067, p=0.048] when all subjects with APOE ε4, who typically show lower CSF Aβ42 levels (16 out of 47) were removed.

Importantly, there was an inverse correlation between CSF A β 42 levels and the score on the HAM-D 17-item scale, both in the whole sample [r=−0.420, p=0.003] and in subjects with MDD only [r=−0.398, p=0.036], indicating that lower CSF levels of A β 42 were associated with more depressive symptoms.

Fifteen subjects with MDD were on antidepressant treatment at the time of test, but no difference was actually observable within the MDD group as a function of taking antidepressants.

Eliminating three significant outliers, F₂-isoprostanes levels differed significantly across condition [t(38)=3.818, p=0.001], with MDD subjects (n=27) having much higher levels than controls (n=13) (Figure, bottom, suggesting increased oxidative stress associated with depression. A β 42 inversely correlated with F₂-isoprostanes levels [r=−0.331, p=0.037], when considering both subjects with MDD and controls.

The reduction in CSF Aβ₄₂ observed in the elderly depressed individuals 60 and older was not influenced by age of onset of first depressive episode. Thus even those with onset of depression at age 40 had a reduction in CSF Aβ₄₂. Another important finding is that many of the depressed individuals with reductions in CSF Aβ₄₂ remained quite depressed despite treatment with adequate doses of currently available conventional antidepressants. This demonstrates that depressive individuals irrespective of age including young depressive individuals have a reduction in CSF Aβ₄₂ that as previously demonstrated reflects increased fibrillar amyloid brain deposits.

### CONCLUSIONS

These results demonstrate that elderly, cognitively intact subjects with MDD, similarly to subjects with AD and MCI, show Aβ₄₂ reductions in CSF. However, unlike AD and MCI sufferers, the MDD subjects do not show differences in CSF tau levels. In addition, subjects with depression also present increased oxidative stress, as measured by F₂-isoprostanes, which were inversely correlated with Aβ₄₂.

Aβ is known to be neurotoxic. Thus, increased brain amyloid load demonstrated by the CSF Aβ₄₂ reductions provides a basis for treating mood disorders such as MDD. Lower CSF Aβ₄₂ levels which are known to reflect greater brain deposits are significantly correlated with a greater number of depressive symptoms both in the depressed group and in controls as measured by scores on the Hamilton depression Scale.

This indicates that brain amyloid deposits cause depressive symptoms and lowering all neurotoxic forms of amyloid β prevents or treats depressive symptoms.

1. A method of diagnosing a mood disorder, such as major depression, minor depression or dysthymia in a subject comprising:
a) collecting a biological test sample selected from the group consisting of whole blood, serum, plasma, urine and cerebrospinal fluid from said subject;
b) determining the level of or concentration of amyloid beta present in the biological test sample;
c) comparing the level of or concentration of amyloid beta in the biological test sample with the level of or concentration of amyloid beta in one or more persons free from a mood disorder such as major depression, minor depression or dysthymia, or with a previously determined reference range for amyloid beta established from subjects free of a mood disorder such as major depression, minor depression or dysthymia, and
d) diagnosing a mood disorder such as major depression, minor depression or dysthymia in the subject if the level of or concentration of amyloid beta is lower in the biological test sample from the subject than the level of or concentration of amyloid beta in one or more persons free from a mood disorder such as major depression, minor depression or dysthymia, or with a previously determined reference range for amyloid beta established from subjects free of a mood disorder such as major depression, minor depression or dysthymia.

2. The method of claim 1, wherein the biological sample is cerebrospinal fluid (CSF).

3. The method of claim 1, wherein decreased concentration or reduced level of or concentration of amyloid beta in a biological test sample correlates with the presence of a mood disorder.

4. The method of claim 1, wherein said amyloid beta is Abeta₄₀ or Abeta₄₂.

5. The method of claim 1, wherein amyloid beta is measured by a quantitative method selected from the group consisting of an immunological or biochemical assay specific for amyloid beta.

6. The method of claim 5, wherein said method is selected from the group consisting of an enzyme-linked immunosorbent assay (ELISA), a Western blot assay, a Northern blot assay, and a Southern blot assay.

7. The method of claim 5, wherein the quantitative method comprises testing at least one aliquot of the test sample, comprising the steps of:
a) contacting the aliquot with an antibody that is immunospecific for amyloid beta;
b) quantitatively measuring any binding that has occurred between the antibody and the test sample.

8. The method of claim 7, wherein the antibody is a monoclonal or polyclonal antibody.

9. The method of claim 7, wherein the step of quantitatively measuring comprises testing a plurality of aliquots with a plurality of antibodies for the quantitative detection of amyloid beta.

10. The method of claim 1 wherein the mood disorder is selected from the group consisting of major depressive disorder, minor depression and dysthymia.

11. The method of claim 1 wherein the mood disorder is major depressive disorder.

12. The method of claim 1 wherein the subject is elderly.

13. The method of claim 1 wherein the subject is not suffering from dementia.

14. A method of identifying a subject suffering from a mood disorder such as major or minor depression or dysthymia that may be successfully treated by an agent that affects amyloid beta levels comprising:
a) collecting a biological test sample selected from the group consisting of whole blood, serum, plasma, urine and cerebrospinal fluid from said subject;
b) determining the level of or concentration of amyloid beta present in the biological test sample;
c) comparing the level of or concentration of amyloid beta in the biological test sample with the level of or concentration of amyloid beta in one or more persons free from a mood disorder such as major depression, minor depression or dysthymia, or with a previously determined reference range for amyloid beta established from subjects free of a mood disorder such as major depression, minor depression or dysthymia, and
d) determining whether the level of or concentration of amyloid beta is lower in the biological test sample from the subject than the level of or concentration of amyloid beta in one or more persons free from a mood disorder such as major depression, minor depression or dysthymia, or with a previously determined reference range for amyloid beta established from subjects free of a mood disorder such as major depression, minor depression or dysthymia.

15. (canceled)

16. (canceled)

17. (canceled)

18. A method of identifying a subject at risk for developing a major or minor depressive episode or disorder comprising:
a) collecting a test sample from said subject;
b) analyzing said test sample for the presence of amyloid beta levels; and
c) comparing the level of or concentration of amyloid beta in the test sample with the level of or concentration of amyloid beta in one or more persons free from a major or minor depressive episode/disorder, or with a previously determined reference range for amyloid beta established from subjects free of a major or minor depressive disorder.

19. (canceled)
